(19) United States
(12) Patent Application Publication
(10) Pub. No.: US 2011/0029000 A1
(43) Pub. Date: Feb. 3, 2011

(54) IN VIVO SUTURE ASSEMBLIES

(75) Inventors: Gilad Lavi, Rishon Lezion (IL); Eli Bar, Moshav Megadim (IL); Gil Griefner, Tel Aviv (IL); Nissim Darvish, Zrufa (IL)

Correspondence Address: The Law Office of Michael E. Kondoudis, 888 16th Street, N.W., Suite 800, Washington, DC, 20006, US

(73) Assignee: BLUE SURGICAL LTD., ZRUFA (IL)

(21) Appl. No.: 12/739,909

(22) PCT Filed: Oct. 26, 2008
(86) PCT No.: PCT/IL08/01409

§ 371 Date: Oct. 6, 2010

Related U.S. Application Data
(62) Division of application No. 61/067,029, filed on Feb. 26, 2008.
Application No. 61/067,029 is a division of application No. 61/133,618, filed on Jul. 2, 2008.
(60) Provisional application No. 60/996,056, filed on Oct. 26, 2007.

(51) Int. Cl. A61B 17/04

(52) U.S. Cl. 606/148

(57) ABSTRACT

Disclosed is a suturing assembly for suturing in vivo tissue. The suturing assembly comprises a first suture thread positioned along a first surface of an in vivo tissue having a first surface and a second surface and at least one suture needle configured to pass through in vivo tissue. The suturing assembly additionally comprises a suture catch located on at least one side of the at least one suture needle, the suture catch configured to releasably catch the suture and draw the suture through the in vivo tissue during a first passage through the in vivo tissue such that the suture is formed into a first loop on a surface of the in vivo tissue.

### RELATED APPLICATIONS

This application claims priority from U.S. Provisional Application Nos. 60/996,056, filed 26 Oct. 2007; 61/067,029, filed 26 Feb. 2008; and 61/133,618, filed 2 Jul. 2008, the contents of all of which are incorporated by reference as if fully set forth herein.

### FIELD AND BACKGROUND OF THE INVENTION

The present invention, in some embodiments thereof, relates to a suturing assembly that stitches in vivo tissue and, more particularly, but not exclusively, to a suturing assembly configured to produce lengths of lock and/or chain stitches through in vivo tissue.

The number of people being overweight or obese has reached truly epidemic proportions in the United States; with 61 percent of the adult population either overweight or obese in 1999. Obesity impacts other diseases. For example, the incidence of type 2 diabetes, a major consequence of obesity, has increased 49 percent from 1990 to 2000.

For someone overweight, losing as little as 5 percent of body weight may lower the risk for several diseases, including heart disease and diabetes. However, to lose weight and keep it off over time, it is not a simple task.

Methods for achieving and maintaining weight loss with minimal suffering are often centered on surgical operations designed to increase feelings of satiation.

Relatively risk-free surgical solutions are usually administered with minimal invasive technique, often through an endoscope. In endoscopic weight loss solutions portions of gastric tissue are often restricted in their maximal expansion, thereby achieving satiation on small food quantities.

Of the endoscopic procedures, plication of unincised, herein intact, gastric tissue appears to have great promise for providing safe gastric restriction as, in general “intraluminal gastroplications [on intact tissue] result in . . . a flat scar . . . ” without the bleeding associated with procedures that incise the gastric tissue.

“Understanding Endoluminal Gastroplications: A Histopathologic Analysis Of Intraluminal Suture Plications”, A. Feitoza et al, Gastrointestinal Endoscopy, Volume 57, Issue 7, 2003, Pages 868-876.

Additionally, plication that relies on continuous suture installs faster than interrupted sutures, possibly resulting in less morbidity.

Additional background art includes the following patents, the contents of all of which are incorporated by reference as if fully set forth herein:

U.S. Pat. No. 7,220,266 (Gambale);

US Patent Publication Numbers:

US 2006/0282089 (Stokes et al);

US 2006/0224184 (Stefanchik et al);

US 2006/0206119 (Chu); and

US 2005/0203548 (Weller et al); and

### WIPO Patent Publication Numbers:

WO 054667 (Stefanchik et al);

WO 05112786, (Nobis et al); and

WO2007127774 (Sauer).

### SUMMARY OF THE INVENTION

According to an aspect of some embodiments of the invention, there is provided a suturing assembly for suturing in vivo tissue. The suturing assembly comprises a first suture thread positioned along a first surface of an in vivo tissue having a first surface and a second surface and at least one suture needle configured to pass through in vivo tissue. The suturing assembly additionally comprises a suture catch located on at least one side of the at least one suture needle, the suture catch configured to releasably catch the suture and draw the suture through the in vivo tissue during a first passage through the in vivo tissue such that the suture is formed into a first loop on a surface of the in vivo tissue.

According to some embodiments of the invention, the at least one suture needle is additionally configured to pass through the first loop during a second passage through the in vivo tissue and draw the suture through into a second loop through the first loop.

According to some embodiments of the invention, the at least one suture needle comprises at least two suture needles: at least one first suture needle spaced a distance from at least one second suture needle.

According to some embodiments of the invention, at least one of: the at least one first suture needle, and the at least one second suture needle, are curved.

According to some embodiments of the invention, at least one of: the at least one first suture needle, and the at least one second suture needle, are straight.

According to some embodiments of the invention, the assembly includes one cutting element configured to cut the in vivo tissue between the first loop and the second loop created by the at least one first suture needle spaced a distance from at least one second suture needle.

According to some embodiments of the invention, the one cutting element cuts the in vivo tissue during at least one of: following formation of the first loop and the second loop, and during formation of the first loop and the second loop.

According to some embodiments of the invention, the cutting element is operatively associated with a cutting element actuator configured to cause the cutting element to move and pierce the in vivo tissue prior to the cutting.

According to some embodiments of the invention, the assembly includes one elongate plate having a surface configured for pressing against an in vivo tissue, the one elongate plate having at least two apertures: at least one first aperture configured to allow passage of the at least one first suture needle, and at least one second aperture configured to allow passage of the at least one second suture needle.

According to some embodiments of the invention, the assembly includes one elongate plate having a surface configured for pressing against an in vivo tissue, the one elongate plate having at least one aperture configured to allow passage of the at least two suture needles therethrough.

According to some embodiments of the invention, the first loop and the second loop are formed proximate to the first surface of the in vivo tissue.

According to some embodiments of the invention, the first loop and the second loop are formed proximate to the second surface of the in vivo tissue.

According to some embodiments of the invention, the first loop and the second loop each include an inner boundary defining a space having at least two areas: a first area sufficiently large to allow passage of the at least one suture needle therethrough during the second pass, and a second area sufficiently small to compress a portion of the first loop.

According to some embodiments of the invention, the assembly includes a hook operatively associated with the second surface and configured to releasable grasp and expand: the first loop from the second area to the first area, and the second loop from the second area to the first area.

According to some embodiments of the invention, the first loop and the second loop are formed proximate to the first surface of the in vivo tissue and the assembly includes, proximate to the first surface, a second suture operatively associated with a shuttle.

According to some embodiments of the invention, the first loop and the second loop each include an inner boundary defining a space having at least two areas: a first area sufficiently large to allow passage of the shuttle and shuttle suture therethrough, and a second area sufficiently small to compress a portion of the shuttle suture against the first surface.

According to some embodiments of the invention, wherein the at least one suture needle is slidingly connected to a first elongate plate operatively associated with the first surface of the in vivo tissue.

According to some embodiments of the invention, the assembly includes at least one needle advancer configured to advance the at least one suture needle along the elongate plate following forming of the first loop.

According to some embodiments of the invention, the assembly includes a second plate operatively associated with the second surface of the in vivo tissue.

According to some embodiments of the invention, the first plate includes at least one first elongate plate aperture and the second plate includes at least one second elongate plate aperture, the at least one second elongate plate aperture being substantially aligned with the at least one first elongate plate aperture.

According to some embodiments of the invention, at least one of the first plate and the second plate includes a compression mechanism configured to reduce the distance between the plates and press the in vivo tissue therebetween.

According to some embodiments of the invention, wherein the at least one suture needle is rotatably connected to an elongate plate and configured to assume at least two positions: a first position wherein the at least one suture needle is non-perpendicular to the longitudinal axis of the plate, and a second position wherein the at least one suture needle is perpendicular to the longitudinal axis of the plate.

According to some embodiments of the invention, the assembly includes at least one needle positioner configured to maintain the at least one suture needle in the second position during at least one passage through the in vivo tissue.

According to some embodiments of the invention, the at least one suture needle is operatively associated with at least one needle mover configured to actuate linear movement of the at least one suture needle along an operatively associated elongate plate.

According to some embodiments of the invention, the at least one suture needle is operatively associated with at least one needle actuator configured to cause the at least one suture needle, when the at least one suture needle is in the second position, to move in two directions perpendicular to an operatively associated elongate plate.

According to some embodiments of the invention, the at least one needle actuator comprises a cord.

According to some embodiments of the invention, the assembly includes a cutting element configured to cut the in vivo tissue during at least one of: following formation of the first loop and the second loop, and during formation of the first loop and the second loop.

According to some embodiments of the invention, the cutting element is operatively associated with a cutting element actuator configured to cause the cutting element to move and pierce the in vivo tissue prior to the cutting.

According to some embodiments of the invention, the assembly includes one elongate plate having a surface configured for pressing against an in vivo tissue, the one elongate plate having at least one aperture configured to allow passage of the at least one suture needle therethrough.

According to some embodiments of the invention, the at least one aperture comprises at least two apertures, at least one first aperture spaced a distance from at least one second aperture.

According to some embodiments of the invention, an axis passing through the at least first one aperture and the at least one second aperture is parallel to the longitudinal axis of the one elongate plate.

According to another aspect of some embodiments of the invention, there is provided a lock stitch suturing assembly for suturing in vivo tissue, the lock stitch assembly comprising: a needle suture positioned proximate to a second surface of an in vivo tissue, at least one suture needle having a suture catch, the at least one suture needle configured to: pass through a first surface and the second surface of the vivo tissue, catch the needle suture on the suture catch, and draw the needle suture through the in vivo tissue thereby forming a loop projecting beyond the first surface of the in vivo tissue, and a shuttle connected to a shuttle suture and configured to pass through the loop, thereby forming a lock stitch following tightening of the loop around the shuttle suture.

According to still another aspect of some embodiments of the invention, there is provided a chain stitch suturing assembly for suturing in vivo tissue, the chain stitch suturing assembly comprising: a first suture positioned proximate to a first surface of an in vivo tissue, at least one suture needle having a suture catch, the at least one suture needle configured to: catch the first suture on the suture catch, and pass a first pass through the first surface and a second surface of the vivo tissue, and a hook operatively associated on the second surface and configured to releasably pull the first suture into a loop that radially outwardly extends along the second surface from the at least one suture needle, the loop configured with an inner boundary of a size that is larger than the outside diameter of the at least one suture needle.

According to a further aspect of some embodiments of the invention, there is provided a suturing assembly for suturing in vivo tissue, the suturing assembly comprising: an elongate plate having at least one aperture configured to allow passage of a needle, at least one suture needle rotatably operatively associated on the elongate plate, the at least one suture needle being rotatable to at least two positions with respect to the elongate plate: a first position wherein the at least one suture needle is non-perpendicular to the longitudinal axis of the elongate plate, and a second position wherein the at least one suture needle is perpendicular to the longitudinal axis of the plate, and a suture catch projecting radially outward from at least one side of at least one suture needle, the suture catch including an opening configured to releasably catch a portion of a suture when the at least one suture needle is in the second position and passes a first pass through the in vivo tissue.

According to some embodiments of the invention, when the at least one suture needle passes through the in vivo tissue, the suture catch forms the suture into a first loop extending from a first surface of the in vivo tissue.

According to some embodiments of the invention, upon passing a second pass through the in vivo tissue, the suture catch forms a second loop extending through the first loop.

According to some embodiments of the invention, the assembly includes, proximate to the first surface, a second suture operatively associated with a shuttle, the shuttle and the at least one suture needle configured to pass through the first loop, thereby forming a lock stitch in the in vivo tissue.

According to some embodiments of the invention, upon the at least one suture needle passing the first pass, the suture catch forms the suture into a first loop extending from a second surface of the in vivo tissue.

According to some embodiments of the invention, upon passing a second pass through the in vivo tissue, the suture catch forms a second loop extending through the first loop.

According to a still further aspect of some embodiments of the invention, there is provided a suturing assembly for suturing in vivo tissue, the suturing assembly comprising: one elongate plate having a surface configured for pressing against an in vivo tissue, the one elongate plate having at least one aperture configured to allow passage therethrough of at least two suture needles located a distance from each other, at least one first suture needle connected to a first suture, the first needle configured to pass multiple times through the at least one aperture and create a first length of stitches in the in vivo tissue, at least one second suture needle located a distance from the one first suture needle and configured to pass multiple passes through the at least one aperture and create a second length of stitches in the in vivo tissue.

According to some embodiments of the invention, at least one of: the at least one first suture needle, and the at least one second suture needle, are curved.

According to some embodiments of the invention, at least one of: the at least one first suture needle, and the at least one second suture needle, are straight.

According to some embodiments of the invention, the first length of stitches is substantially parallel to the second length of stitches.

According to some embodiments of the invention, the first length of stitches is formed simultaneously with formation of the second length of stitches.

According to some embodiments of the invention, the assembly includes a cutting element configured to cut the in vivo tissue between the first length of stitches and the second length of stitches.

According to some embodiments of the invention, the cutter cuts the in vivo tissue during at least one of: following formation of the first and second lengths of stitches, and during formation of the first and second lengths of stitches.

According to an additional aspect of some embodiments of the invention, there is provided a suturing assembly for suturing in vivo tissue, the suturing assembly comprising: an elongate plate having at least one aperture configured to allow passage of at least one suture needle, at least one suture needle configured to pass through the aperture and an in vivo portion of tissue when the at least one suture needle is perpendicular to the elongate plate, and a suture catch on the at least one suture needle configured for releasably catching a portion of a suture during the passage of the at least one suture needle.

According to another additional aspect of some embodiments of the invention, there is provided a suturing assembly for suturing in vivo tissue, the suturing assembly comprising: an elongate plate configured for contacting an in vivo tissue, at least one suture needle moveably connected to the elongate plate, at least one suture needle moving assembly operatively associated with the at least one suture needle and configured to move the at least one suture needle with respect to the elongate plate, and a suture catch projecting radially outward from at least one side of the at least one suture needle, the suture catch configured to releasably catch the suture.

According to some embodiments of the invention, the at least one suture needle moving assembly includes an actuation mechanism located remotely from at least one suture needle moving assembly.

According to some embodiments of the invention, the suture needle moving assembly is configured to cause the at least one suture needle to move perpendicularly with respect to the elongate plate.

According to some embodiments of the invention, the suture needle moving assembly is configured to cause the at least one suture needle to rotatably move with respect to the elongate plate.

According to some embodiments of the invention, suture needle moving assembly is configured to cause the at least one suture needle to move parallel with respect to the elongate plate.

According to some embodiments of the invention, the suture needle moving assembly is configured to cause the at least one suture needle to move parallel to a longitudinal axis of the elongate plate.

According to another further aspect of some embodiments of the invention, there is provided a method of in vivo suturing, comprising: providing at least one suture needle, providing a suture catch on the at least one suture needle, causing the at least one suture needle to linearly pass a first pass through a first in vivo tissue surface and a second in vivo tissue surface on an in vivo tissue portion, catching a first suture portion on the suture catch, and extending the first suture portion beyond the first in vivo tissue surface in a first loop.

According to some embodiments of the invention, the method additionally includes: causing the at least one suture needle to linearly pass a second pass through the first loop, and passing the at least one suture needle through the first in vivo tissue surface and the second in vivo tissue surface.

According to some embodiments of the invention, the method additionally includes: catching a second suture portion on the suture catch, extending the second suture portion beyond the first in vivo tissue surface in a second loop, and extending the second loop through the first loop.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A shows a tissue suturing assembly configured to produce lock and/or chain stitches through in vivo tissue, according to embodiments of the invention;

FIGS. 1B-5 show a method for preparing tissue for plication using the assembly shown in FIG. 1A, according to embodiments of the invention;

FIGS. 6-16 show perspective views of a needle and needle assemblies while producing chain-linked stitches wherein the chains are located on an upper surface of in vivo tissue, according to some embodiments of the invention;

FIG. 17 shows an isometric view of an in vivo tissue suturing assembly in a compact configuration according to some embodiments of the invention;

FIGS. 18-25 show partial cross-sectional views of the in vivo tissue suturing assembly shown in FIG. 17 during operation, according to some embodiments of the invention;

FIGS. 26-28 show isometric views of the plate and needle assembly of the in vivo tissue suturing assembly shown in FIG. 17, according to some embodiments of the invention;

FIGS. 29-32 show details of the suture feeding system of the in vivo tissue suturing assembly shown in FIG. 17, according to some embodiments of the invention;

FIGS. 33-36 show cross-sectional views of the needle assembly and plicated tissue, according to some embodiments of the invention; and

FIGS. 37-40, and 41-44 show alternative embodiments for needle activation, according to some embodiments of the invention;

FIG. 45 shows a lower surface chain stitch assembly, according to embodiments of the invention;

FIG. 46A shows an isometric view of the suture needle having penetrated the tissue, according to some embodiments of the invention; FIG. 46B shows the embodiment of FIG. 46A after the suture needle moved into the suture loop created by the hook, according to some embodiments of the invention;

FIG. 47 shows the embodiment of FIG. 46B after the hook has moved backwards, according to some embodiments of the invention;

FIG. 48 shows the embodiment of FIG. 47 following retraction of the suture needle, according to some embodiments of the invention;

FIG. 49 shows the embodiment of FIG. 48 after both the suture needle and hook have moved to a pre-stitch position, according to some embodiments of the invention;

FIG. 50 shows the embodiment of FIG. 49 with the suture needle perforating the tissue in synchronization with the hook, according to some embodiments of the invention;

FIG. 51 shows the embodiment of FIG. 50 with the hook rotated to enable release of the suture loop, according to embodiments of the invention;

FIG. 52 shows the embodiment of FIG. 51 with the suture needle moved forward, and where a first loop stitch has been formed, according to embodiments of the invention;

FIG. 53 shows the embodiment of FIG. 52 after the hook has rotated to enable capturing the suture thread of a second loop, according to embodiments of the invention;

FIG. 54A shows the embodiment of FIG. 53 with the hook having moved backwards, thereby pulling the second loop, according to embodiments of the invention;

FIGS. 54B-54D show an embodiment in which the in vivo tissue suturing assembly is contained within an endoscopic tool, according to some embodiments of the invention;

FIG. 55 shows the in vivo tissue suturing assembly with an upper surface lock stitch facilitator, according to embodiments of the invention;

FIG. 56 shows an isometric view of the in vivo tissue suturing assembly and tissue, according to embodiments of the invention;

FIG. 57 shows a cross-section view of the embodiment in FIG. 56, according to embodiments of the invention;

FIG. 58A shows a cross-section of the in vivo tissue suturing assembly and sutures using dual pressure plates on two portions of plicated tissue, according to embodiments of the invention;

FIGS. 58B-58C show cross-section views of two types of stitches sutured while using the plication assembly of FIG. 58A, according to embodiments of the invention;

FIG. 59 shows an isometric view of the plication assembly of FIG. 58A, according to embodiments of the invention;

FIGS. 60A-60B show isometric views of double and single suture thread stitching, respectively, according to embodiments of the invention;

FIG. 61 shows a cross section of a portion of plicated tissue with two rows of suture;

FIG. 62 shows an isometric view of an in vivo tissue suturing assembly equipped with dual suture needles in the open position, according to some embodiments of the invention;

FIG. 63 shows an isometric view of the in vivo tissue suturing assembly of FIG. 62 in a compact configuration, according to some embodiments of the invention;

FIG. 64 shows a side view of the in vivo tissue suturing assembly shown in FIG. 62, according to some embodiments of the invention;

FIGS. 65-66 show frontal views of the in vivo tissue suturing assembly shown in FIG. 63, according to some embodiments of the invention;

FIGS. 67A-67B show side views of the in vivo tissue suturing assembly shown in FIG. 63, according to some embodiments of the invention;

FIGS. 68-69 show front cross-section views of the in vivo tissue suturing assembly shown in FIGS. 65-66 according to some embodiments of the invention;

FIG. 70A shows the stomach in cross-section, according to some embodiments of the invention;

FIG. 70B shows a planned stomach plication procedure, according to some embodiments of the invention;

FIG. 70C shows an exterior view of the stomach following the plication procedure of FIG. 70B, according to some embodiments of the invention;

FIG. 71A-71C show cross-sectional views of the stomach during the plication procedure of FIG. 70B, according to some embodiments of the invention;

FIGS. 72A-72B show in vivo tissue suturing an aerial view of the in vivo tissue suturing assembly of FIG. 71B following plication, according to some embodiments of the invention;

FIG. 72C shows a side view of a portion of the in vivo tissue suturing of FIG. 71C, according to some embodiments of the invention; and

FIGS. 73-74 show an alternative embodiment of the double row suture assembly of FIGS. 68-69, according to some embodiments of the invention.

### DESCRIPTION OF EMBODIMENTS OF THE INVENTION

The present invention, in some embodiments thereof, relates to a suturing assembly that stitches in vivo tissue and, more particularly, but not exclusively, to a suturing assembly configured to produce lengths of lock and/or chain stitches through in vivo tissue.

In embodiments, the present invention comprises a suturing mechanism for suturing in vivo tissue, for example within the lumen of a hollow organ.

More specifically, but not exclusively, the present invention may be used to plicate tissues within the hollow organ. While the description which follows focuses on the plication of tissue within the gastric cavity, the present invention may alternatively have use in suturing tissue in organs that may include, inter alia, the bladder, heart chambers, lungs, gall bladder, intestine, colon, esophagus, and diaphragm.

As used herein, the teem “plication” refers to an operation for reducing the size of a hollow structure by making folds or tucks in its walls. Further, the term “plicated” with reference to tissue, refers to tissue that has been folded.

The present invention includes a handle connected to a plate having two compression plates comprising dual rails or perforations through the plates that are initially substantially opposed in a compact configuration and open, meaning that a distance is created therebetween, to allow introduction of a tissue that has been folded.

Following introduction of the folded tissue, a hand-activated mechanism closes the distance between the two plates, thereby creating pressure on the folded tissue, to compress the walls of the tissue against each other with an optimal pressure that facilitates suturing.

Suturing that occurs following compression of the folded tissue forms a permanently plicated area within the organ, thereby reducing the size of the organ and/or influencing the physiologic interaction of the organ tissue, as explained below.

The assembly by which the present invention sutures the folded tissue includes a suturing assembly operatively associated with, in some embodiments, a straight suture needle which during suturing is positioned along a substantially perpendicular axis with respect to a plate and a first surface of the folded tissue. The suturing typically occurs between two rails extending along the plate.

In some embodiments, the assembly is configured with two straight needles that form two lengths of stitches simultaneously.

The present invention includes multiple configurations for producing chain and/or lock stitches in single or double rows along in vivo tissue; and present but a few of the many possible configurations of the assembly and suture stitch configurations will be described.

### Upper Surface Chain Stitching

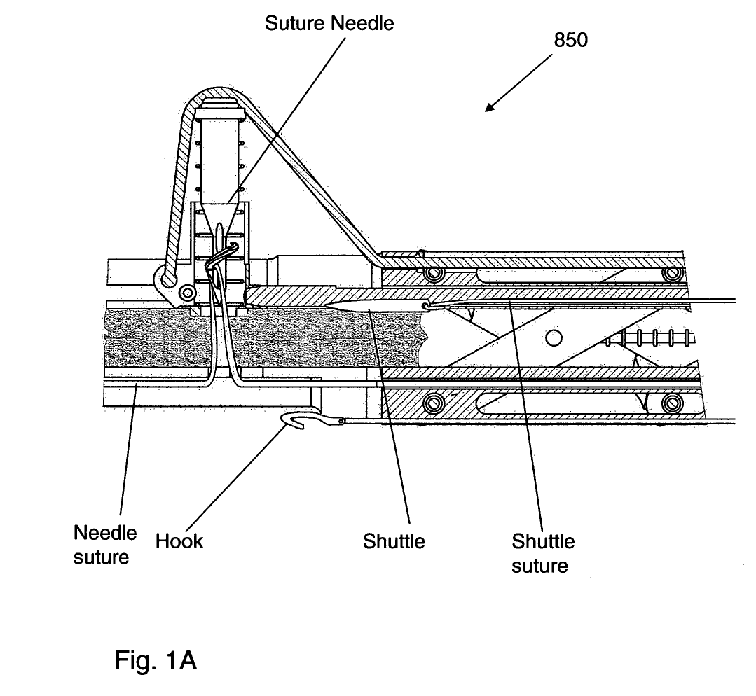

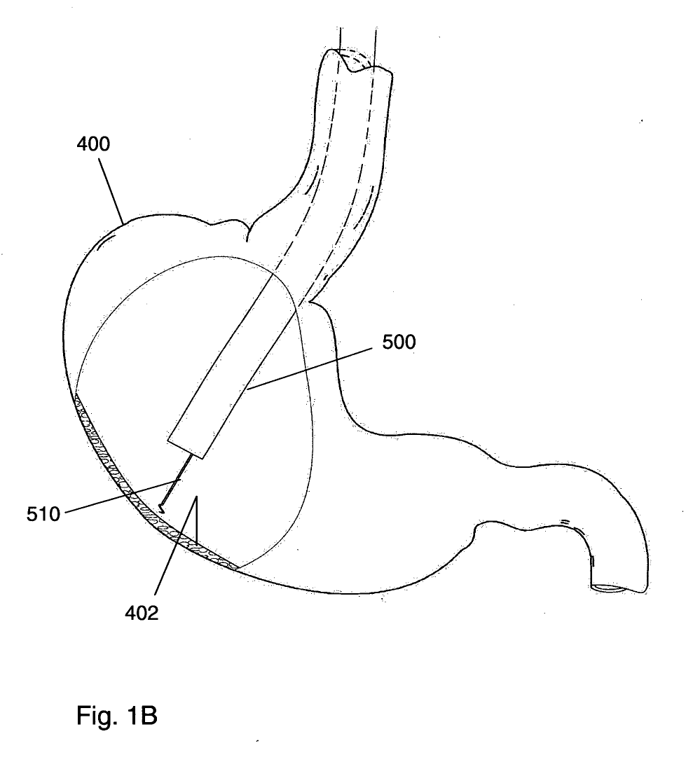

Referring to FIG. 1A, to form a chain stitch with an upper surface chain stitch assembly, a suture needle actuation mechanism causes a suture needle on the suturing assembly to move linearly and perpendicularly into the tissue and out of the tissue; alternatively referred to herein as moving down and up with respect to the plate.

When the suture needle is in the up position, the suture needle is located on a first side of the folded tissue while a suture is positioned on a second side of the folded tissue. A suture needle activating mechanism attached to the suture needle, for example comprising a cable, causes the suture needle to move downward and through the folded tissue, creating a first aperture in the folded portions of tissue. The suture needle passes through the folded tissue until a suture catch, comprising a projection on the suture needle, passes through the second side of the folded tissue and below the suture positioned thereon.

The suture needle is then pulled upward. Alternatively, a spring mechanism on the suture needle assembly causes the suture needle to move upward as an operator relaxes pressure on the suture needle activating mechanism. As the suture needle moves upward, the upper surface of the suture catch contacts the lower surface of the suture and pulls the suture into the aperture in the tissue.

In still further embodiments of the invention, a hydraulic-activated assembly or pneumatic-activated assembly causes movement of the suture needle through the tissue.

The upward movement of the suture needle causes a first loop to form in the suture on the upper side of the tissue.

A traversing mechanism on the suture needle assembly, for example along the two plates, causes the suture needle to move along the two plates, away from the handle, alternatively referred to as moving forward along the two plates.

During the forward motion of the suture needle, the suture needle pulls the first loop forward along the first side of the folded tissue; until the first loop extends a predetermined distance forward from the first aperture through the folded tissue.

Upon reaching the predetermined distance, the suture needle moves downward through the tissue to form a second aperture in the tissue. As the suture catch is below the suture, the suture needle catch moves through the second aperture. The first suture loop remains on the first side of the tissue, with the loop surrounding the second aperture, while the suture needle passes through the tissue.

The suture needle moves downward until the suture catch again moves below the suture positioned on the second side of the folded tissue. The suture needle's direction is then reversed so that the suture catch pulls the suture into the second aperture in the tissue, and forms a second loop of suture extending above the first side of the folded tissue.

The forward motion of the suture needle, followed by creating an additional aperture in the tissue through downward motion of the suture needle, and, thereafter extending another loop above the first surface of the folded tissue, is continued until a series of loops are created in the folded tissue.

In embodiments, the suture needle is activated, for example, by a cable that when drawn tight, causes the suture needle to move downward through the tissue layers and, upon release of the cable, the suture needle returns to an original position above, herein upward, of the tissue layers. The folded tissue is then compressed into a plicated configuration, after which the suture is secured in any one of a plurality of techniques including knotting and/or fusion of strands. Suture fusion includes, for example, ultrasonically welding multifilament suture sections as described in US Patent Application 20050209639, “Fused suture knot” (Gidwani et al), the disclosure of which is hereby incorporated as if fully set forth herein.

### Lower Surface Chain Stitching

The suturing assembly optionally additionally includes a hook located on the second side of the tissue which the operator activates to produce a chain stitch in which the interlinked loops are formed on the lower side of the tissue.

In this embodiment, as the suture needle makes a first pass through the lower side of the tissue, the hook mechanism hooks onto the suture associated with the suture needle and pulls the suture needle suture a distance, along the lower surface of the tissue.

The suture needle then moves upward to return to the first side of the tissue, with the suture captured by the hook mechanism, so that the needle's suture is formed into a first loop along the lower surface.

The suture needle then moves forward along the plates as described above, and makes a second pass through the tissue. When the suture needle and its associated suture are located on the lower side of the tissue, the suture needle is configured to pass through the first loop of suture along the lower surface. The hook mechanism hooks onto the suture associated with the second pass and pulls the needle's suture a distance, along the lower surface of the tissue, thereby being encircled by the first suture pass that formed the first loop while simultaneously locking the first loop, as will be explained below.

Additional passes by the suture needle through the tissue results in a length of chain stitches on the lower tissue surface.

As noted above, the present invention can be configured for creating lock stitches; the following describing but one such embodiment.

### Upper Surface Lock Stitching

The suturing assembly optionally additionally includes a shuttle attached to a shuttle suture, located on the first side of the tissue which the operator activates to produce a lock stitch in which the lock stitches are formed on the upper side of the tissue.

In this embodiment, the suture needle makes a first pass through the tissue, forming a first loop on the upper side of the tissue. The shuttle then passes through the first loop so that the shuttle suture is interposed between the first loop and the upper tissue surface

The suture needle then moves forward along the plates, as described above, and makes a second pass through the tissue. When the suture needle and its associated suture is formed into a second loop on the upper side of the tissue, the shuttle passes through the second loop so that the shuttle suture is interposed between the second loop and the upper tissue surface.

Additional passes by the suture needle through the tissue with passage of the shuttle through each additional loop, results in a length of lock stitches along the upper tissue surface.

### Assembly Insertion Configuration

In embodiments, the suture needle assembly includes a rotational mechanism that:
i) rotates the suture needle from, or to, a position that is substantially perpendicular to the plate in preparation for downward motion; and/or
ii) rotates the suture needle from, or to, a position that is substantially parallel to the plate in preparation for inserting and/or removing the present invention from the hollow organ.

When the suture needle is substantially parallel to the plate, the above-noted plates compress to form a compact configuration that allows easy insertion into a hollow organ, with significantly minimal rubbing against tissue that might result in tissue irritation.

In embodiments, the present invention may be inserted into a hollow organ through a lumen that has a diameter of more than about 2.5, 3.0, 3.5, 4.0, and 5.0 millimeters. Alternatively, the lumen has a diameter of less than about 14.0, 12.0, 10.0, 8.0 and 6.0 millimeters. Optionally, the present invention is used in lumens having a diameter of 3.75 millimeters or more.

### Additional Stitching Configurations

The present invention can be used to provide plication of tissue with aggregate lengths of suture loops of above about 1.0 millimeter to lengths below about 50 millimeters. In embodiments, the pitch of the suture, meaning the length of suture between vertical stitching, can be varied such that within a given length of suturing, for example within the above-noted 50 millimeters, the number of stitches, meaning the substantially vertical placement of suture, can be distanced a minimum of below about 1.0 millimeter and above a maximum of about 20 millimeters.

Optionally, the plication is accomplished during a single insertion of the present invention into the hollow organ. Additionally or alternatively, the present invention can be used to provide multiple areas of plication, for example a first area of plication in the stomach antrum, a second area of plication in the body of the stomach, and a third area of plication near the duodenum.

In further embodiments of the present invention, the suture used has a diameter of more than about 0.5 millimeters or less than about 18 millimeters.

In still further embodiments, the distance between loops, the angle of the aperture through the tissue, the length of the suture line, and/or the number of suture lines, can be varied by changing any one of a number of parameters. For example, the length of each of the loops can be increased or decreased by changing the predetermined distance that the suture needle travels forward, or the loop is pulled forward, from the above-noted aperture in the tissue. Alternatively, repositioning the plates a distance from the first suture line allows the operator to create a second plication that is substantially parallel to the first plication.

In embodiments, the length of the loop, herein referred to as loop pitch, may be as little as 0.5 mm to 5 mm, or as much as 10 mm or more.

### Tissue and Stitch Placement

In embodiments, the present invention is used in conjunction with standard endoscopes; the term endoscopes referring to fiber optic visualization instruments that are used to visualize the inside of hollow organs, including inter alia:

the stomach, the device being referred to as a gastrocsope;

the colon, the device being referred to as a colonoscope; and/or

intra-articular joints, the device being referred to as an arthroscope.

In embodiments, the present invention has a variety of applications related to, inter alia, stomach volume reduction, closure of bleeding tissue, perforation, or ulcers, within a hollow organ. Additionally, the present invention may have use in removal of polyps or nodes along the digestive tract.

In some embodiments, the plication may be positioned by the present invention to additionally or alternatively place pressure upon hunger receptors to reduce or prevent the firing of the hunger receptors, as described in PCT application IL/060048, now published as WO 2006/111961, the contents of which is incorporated by reference as if fully set forth herein.

Alternatively, in some embodiments the plication may be positioned by the present invention to additionally or alternatively:

place pressure upon; and/or

increase the firing of satiety receptors.

Methods and devices for influencing satiety and/or hunger receptors, for example through tissue pressure, are discussed in World Patent Application Number WO2006111961 “Methods And Devices For Limiting Gastric Expansion” (Bar and Darvish).

### Hemostasis

As noted above, the folded tissue is introduced between the two plates which are initially spaced apart to allow for tissue introduction. Optionally, each wall of the introduced tissue has a thickness of more than about 1.0, 1.5, or 2.5 millimeters. Alternatively, each wall of the introduced tissue has a thickness of less than about 6.0, 5.0, 4.0, and 3.0 millimeters.

In some embodiments, the introduced tissue, following compression of the tissue by the instant invention, has a wall thickness of more than about 1.0, 1.5, or 2.5 millimeters or less than about 6.0, 5.0, 4.0, and 3.0 millimeters.

As further noted above, following introduction of the folded tissue between the plates, the distance between the two plates is reduced, thereby creating pressure on the folded tissue, to compress the walls of the tissue against each other with an optimal pressure that facilitates suturing. Such optimal pressure, for example, ensures that the folded tissue can remain properly approximated during the procedure and/or that the folded tissue is properly taut to allow easy passage of the suture needle.

Additionally or alternatively, such optimal pressure ensures that blood has been substantially exsanguinated from the tissue, thereby ensuring a substantially bloodless field, herein hemostasis. As used herein, the term “hemostasis” refers to partial or full “stoppage of blood flow through a blood vessel or organ of the body.” Webster's New World Medical Dictionary, 3rd edition (May, 2008) Wiley Publishing, Inc.

A hemostatically controlled field helps to ensure that blood does not collect in hollow lumens, for example the abdomen, which could serve as a focal point for foreign body reaction and/or infection. Further, a bloodless field ensures that the above-noted endoscope has a clear view of the present invention, without being obscured by blood, to ensure that the suture loops are placed in the proper location and/or with the proper pitch.

In further embodiments, the plates that press folded tissue are equipped with pressure-sensitive mechanisms that ensure that the tissue is exsanguinated without damage to adjacent tissue.

In general, the instant invention allows multiple suture stitches to be applied in a single row and/or during a single insertion of the tool. Additionally, as the tool provides continuous linking of loops, the present invention does not require location of a suture end following a first stitch in order to begin making a second stitch, thereby providing an easy method of creating continuous suturing of plications.

In further embodiments of the invention, the change of direction of the first loop along the first side of the plication perhaps serves to limit tension imparted to the second loop, thereby limiting the tendency of the tissue to pucker, as might be the case in continuous suturing of a single suture fiber. Tissue puckering is to be avoided to prevent unwanted adhesions to occur along a given line of suture and to minimize size, and/or to improve the homogeneity of the created scar.

Moreover, the compact packaging of the suture needle assembly parallel to the plates, as well as the compact configuration of the plates prior to deployment, allow the suturing tool to be deployed in a variety of small lumens, for example the bladder neck, without causing tissue damage.

### Double Row Suturing Assembly

According to some embodiments of the present invention, a double row suturing assembly is used for decreasing the stomach interior volume by reshaping it using a double row of suture stitches and cutting the gastric tissue therebetween possibly optimizing hemostasis and/or accelerating healing.

The double row suturing assembly includes a set of pressure plates that press the plicated gastric walls while the stitches and incision are being made. Natural healing results in permanent tissue connections along the cut areas of the gastric tissue.

The double row suturing assembly performs, inter alia, three functions comprising forward movement, suturing, and cutting; and includes mechanisms to enable advancing the suture needle, suturing, and cutting of the attached tissues.

Applying pressure with the plates, moving the suture needle, and forming the suture stitches, are optionally externally controlled by the operator. In embodiments, the cutting blade is part of the double row suturing assembly such that the gastric tissue is cut as the suture needle moves forward.

Alternatively, following the formation of the suture rows, the cutting blade is activated to move forward, optionally along the pressure plates, and cut the tissue.

The pressure plates of the double row suturing assembly apply pressure on either side of the gastric tissue therebetween while the suturing is being performed. The pressure plates preferably each include an offset of spatially matching guide holes through which the suturing needle is passed during the suturing process. The plates are optionally aligned with the holes opposite each other, so that the suture needle or needles will easily pass through opposing holes. The pressure plates are optionally connected by a compression mechanism such that their mutual position is known and can be predefined.

The clearance between the hole size and the suture needle diameter optionally compensates for any potential misalignment such that the suture needle finds its way in and out, and the stitch is performed at its desired quality.

In embodiments, the holes in each pressure plate are connected by passages, comprising for example slits, so that the suturing thread on both sides of the gastric tissue can be drawn tight with the pressure plates still in position, thereby maintaining pressure on the plication. Once the entire length of suturing has been completed, the pressure plates are moved, leaving the completed stitches with optimal tension.

Suture needle diameter may be between about 0.2 millimeters and 2.0 millimeters and a portion of the suture needle is optionally attached to suturing wire rolled on an external bobbin. The suture thread may be between about 0.1 millimeters and 1.0 millimeters in diameter. In embodiments, the suture needle includes a suture catching mechanism which will be explained below. Additionally, the stitch may be made from one suture thread or multiple parallel suture threads. The guide holes in the pressure plates lead the suture needle during upward and downward movement while establishing a preset perforation pitch.

In embodiments, the suture needle passes a single stitch through a first set of holes in the pressure plates and tissue; moves forward to its next position and passes another single stitch through a second set of holes in the pressure plates and tissue.

The pitch of the stitches is optionally dictated by the distance between the predefined holes in the plate. Optionally, the assembly comes in a kit having several sets of plates, each set configured to provide a different stitch pitch.

In alternative embodiments, the plates include elongate apertures having widths that allow the suture needle to slide unimpeded in either a forward or backward direction; allowing the surgeon to optionally control the stitch pitch, for example, by varying the axial interval at which the needle moves during stitching.

The operator controls the navigation of the tool within the stomach and the position and length of the plication.

According to a further embodiment of the present invention, the plates can be configured with wide passages through which the suture needle passes without the provision of holes.

The double row suturing assembly is optionally implemented endoscopically and, in some embodiments, the assembly is designed to be passed through a standard, off-the-shelf, guiding catheter commonly used for gastric applications.

Alternatively, the assembly is configured with an ergonomic invention-specific catheter that optionally incorporates an optic fiber for visualizing the position of the assembly in the gastric cavity. Alternatively, the assembly incorporates a navigating system that enables the assembly to be brought into the desired location.

Additionally, in embodiments, the assembly includes a tissue grasping system that enables the plication to be grasped and pulled into the active area between the pressure plates. In still further embodiments, the assembly incorporates an expansion system that enables the assembly to be inserted into the endoscope working channel in a collapsed position, expand to a deployed position, and plicate two portions of the gastric tissue, with a first portion of gastric tissue located a distance from a second portion of gastric tissue.

In still additional embodiments, the assembly includes a visual system that enables real time visualization of the plication, suturing and cutting processes.

Although the double row suturing assembly is described for use in internal gastric suturing, it is to be understood that the invention can be configured for a variety of applications in which internal plication suturing is to be performed with minimally invasive access to the site.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. Additionally, the invention is capable of other embodiments or of being practiced or carried out in various ways.

To simplify the description, each embodiment of the in vivo suturing assembly will be shown only with mechanisms that are used during operation while creating a specific stitch length.

Referring now to the drawings:

For purposes of better understanding embodiments of the present invention, as illustrated in FIGS. 1B-4 of the drawings, a method for preparing a plication in gastric tissue is shown.

In FIG. 1B an endoscopic hook 510 is inserted into a stomach 400 via a catheter 500, using a standard endoscope, to approach a portion of inner stomach wall 402. As shown in FIG. 2, endoscopic hook 510 creates a fold 404 in the tissue, alternatively referred to as plication 404. Hook 510 is pulled back by reversing the motion of catheter 500 and/or hook 510.

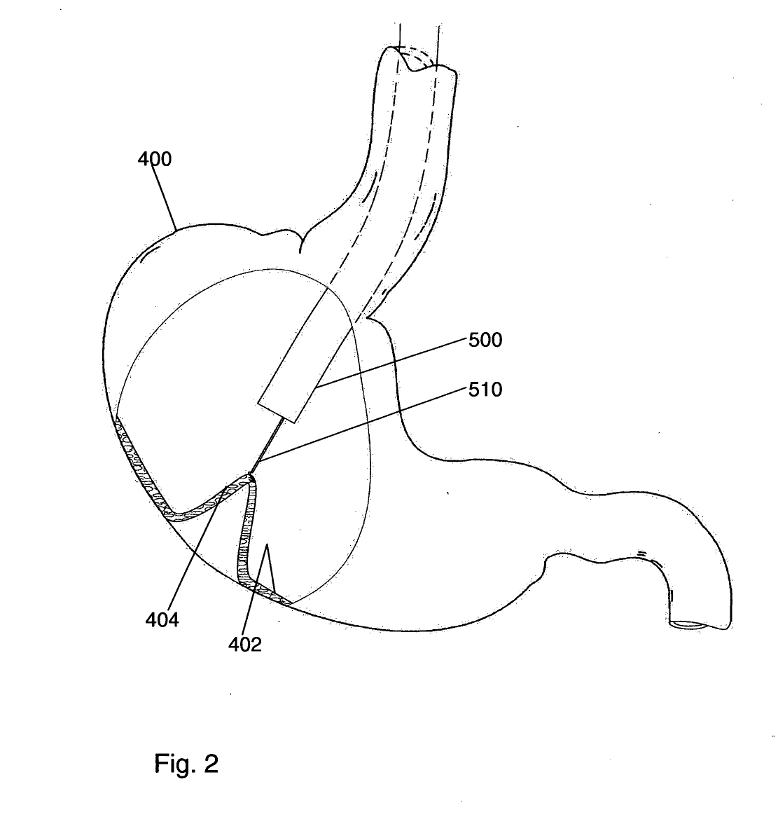

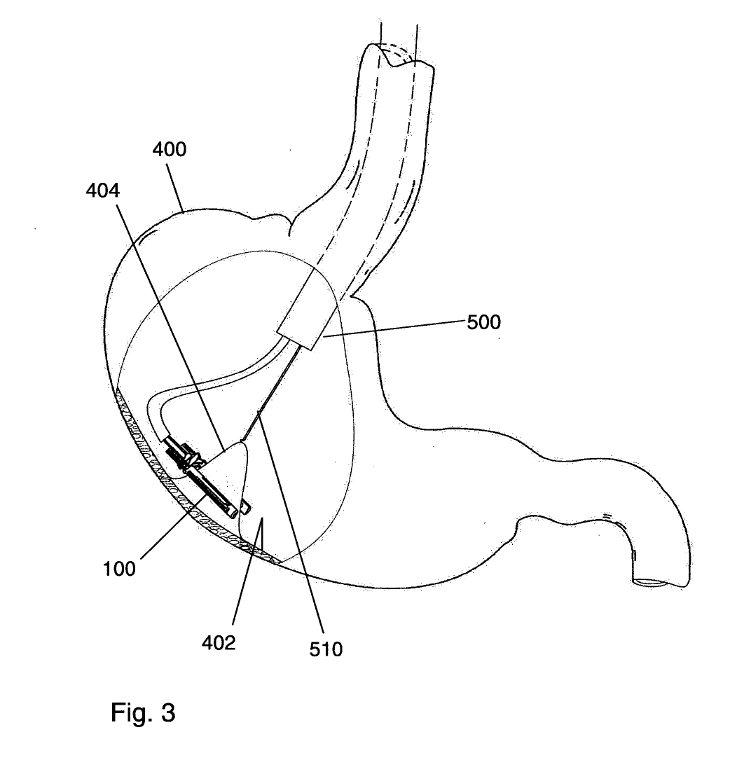

FIG. 3 shows the introduction of a prior art suturing clamp 100 to stabilize fold 404 during suturing. Stabilization of tissue fold 404 is typically viewed through an endoscope (not shown) associated with catheter 500.

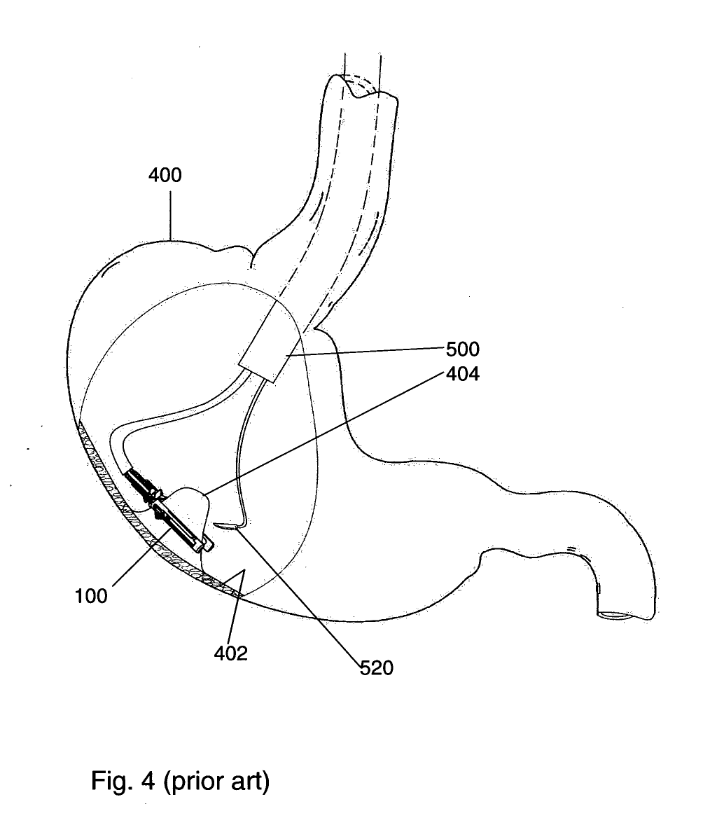

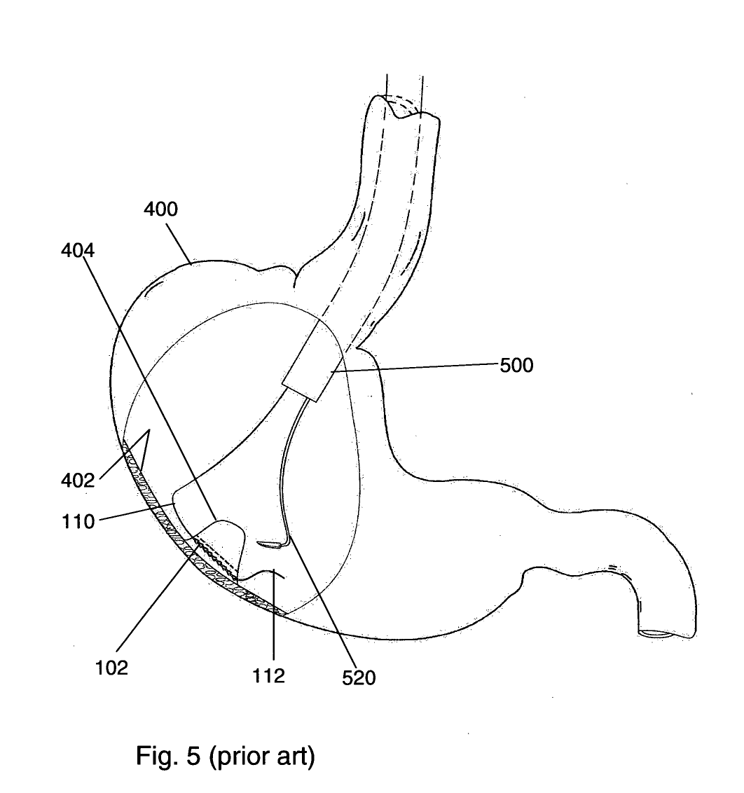

As seen in an existing plication method of FIGS. 4-5, the operator manipulates a suturing tool 520 to apply one or more suture stitches and, in some prior art noted above, create a securing knot following each suture pass. In some prior art, stitches are created in a continuous manner using a single thread passing through the tissue without interposing knots between each stitch.

Following completion of a line of stitches 102 to secure the folded tissue into a plication (FIG. 5), a distal suture 112 is secured and cut, and a proximal suture 110 is cut. The cut portions of distal suture 112 and proximal suture 110 are then removed from stomach 400.

### Upper Surface Chain Stitching

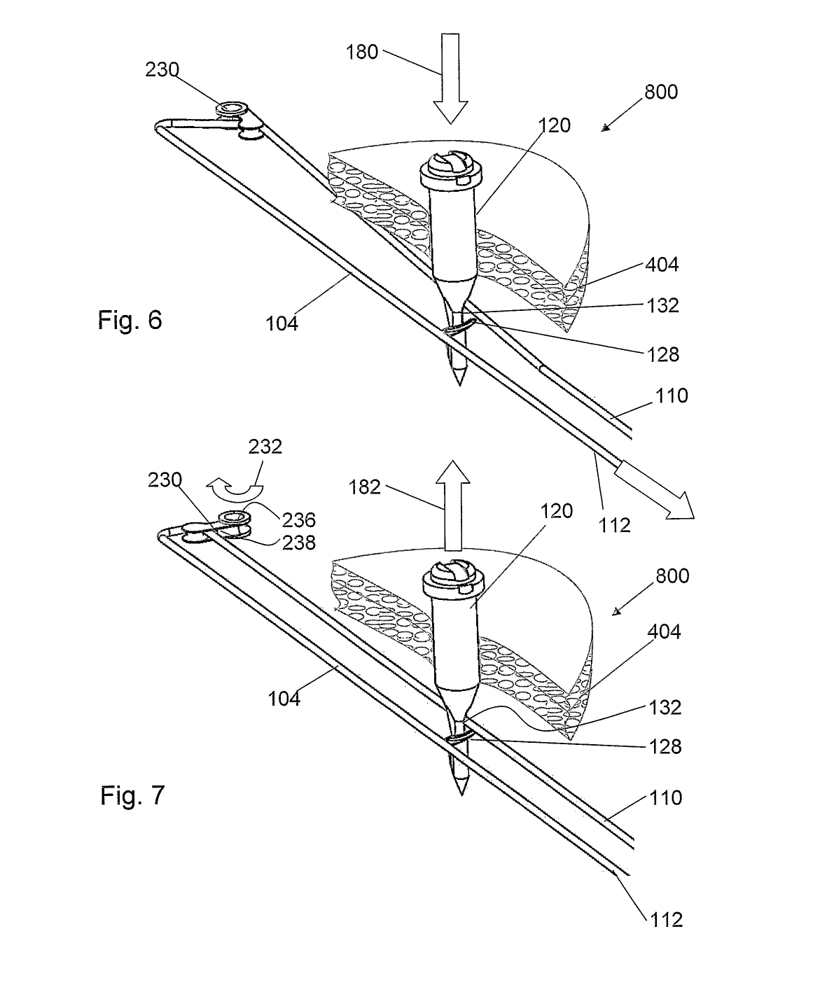

FIGS. 6 to 16 show a portion of a suturing assembly 800 of the instant invention which produces interlinked chains of stitches wherein the linkage between the stitches is located on an upper surface of in vivo tissue.

Suturing assembly 800 comprises a suture needle 120 that has moved in a downward direction 180 to penetrate stomach plication 404 while a suture feeding lever 230 maintains tension along a suture 104.

Optionally, suture feeding lever 230 is spring loaded and rotates around a pivot 236 in a direction 232 to maintain tension on suture 104 which passes through a suture feed aperture 238.

Following penetration, a suture catch 128 catches a portion of suture 104 and is pulled in an upward direction 182. Suture feeding lever 230 rotates in direction 232.

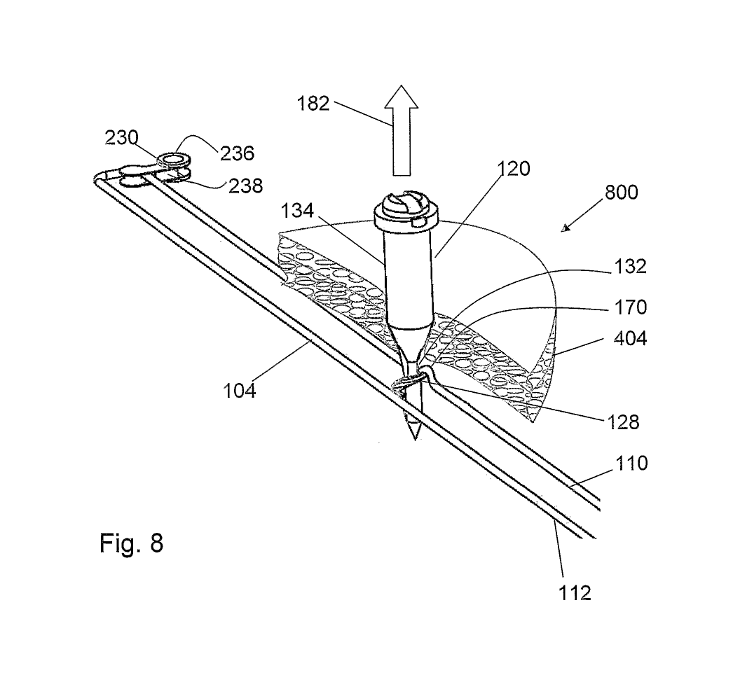

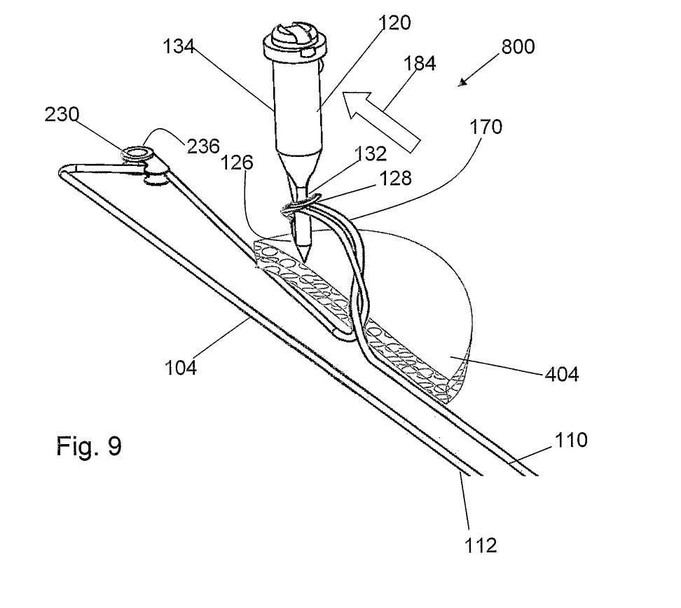

As seen in FIG. 8, suture 104 forms a first suture loop 170 as suture catch 128 moves in direction 182. As seen in FIG. 9, first suture loop 170 has been pulled through plication 404 and suture needle 120 moves forward in a direction 184, pulling first suture loop 170 in forward direction 184.

In embodiments, the forward distance in direction 184 of suture needle 120, hence the length of each loop 170, is preset into suturing assembly 800, for example at a distance of about 3 to 5 millimeters, with mechanisms that will be explained below.

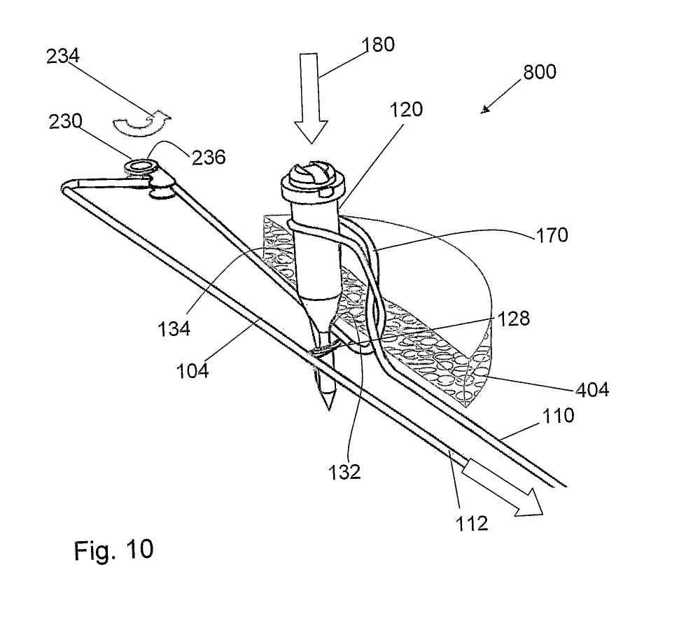

As seen in FIG. 10, suture needle 120 then moves in downward direction 180 through first suture loop 170 to penetrate plication at a second forward position. In embodiments, suture needle 120 includes a groove 132 that aids in positioning suture 104 and maintaining a compact configuration of suture loop 170 during downward movement 180.

Additionally, suture feeding lever 230 has released to rotate in a direction 234 to again maintain tension on suture 104 and to keep it aside for safe penetration of suture needle 120 in forming the next loop 170.

After passing through plication 404, suture catch 128 again engages suture 104.

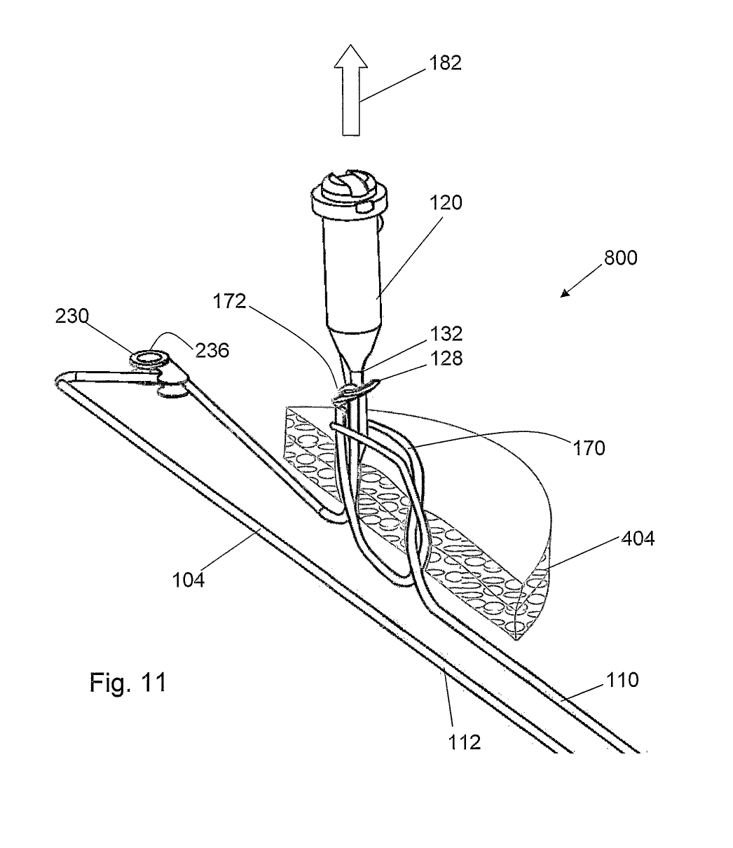

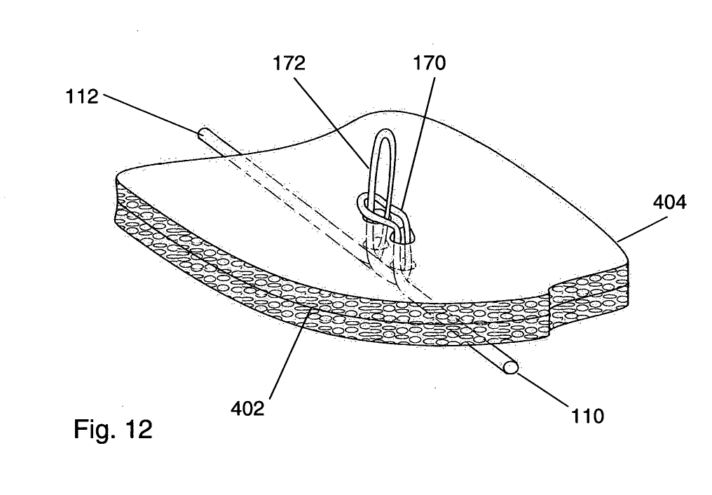

As seen in FIG. 11, suture needle 120 again moves in upward direction 182, thereby creating a subsequent suture loop 172 that is drawn through first suture loop 170; shown in detail in FIG. 12.

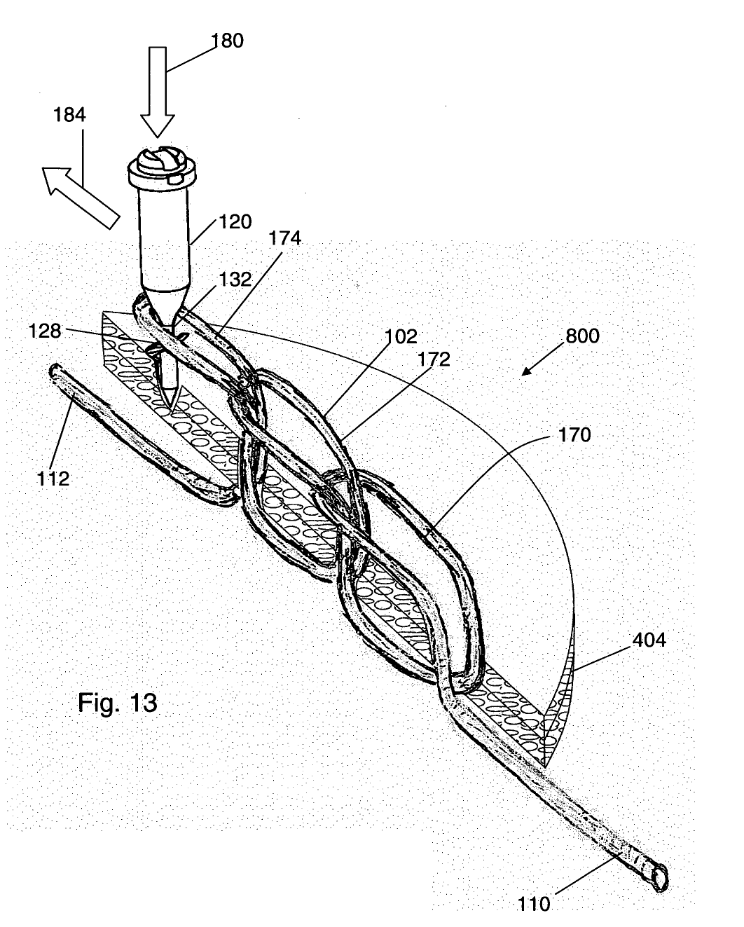

The process is repeated and, as seen in FIG. 13, a third suture loop 174 is passed through second suture loop 172 as suture needle 120 passes in forward direction 184.

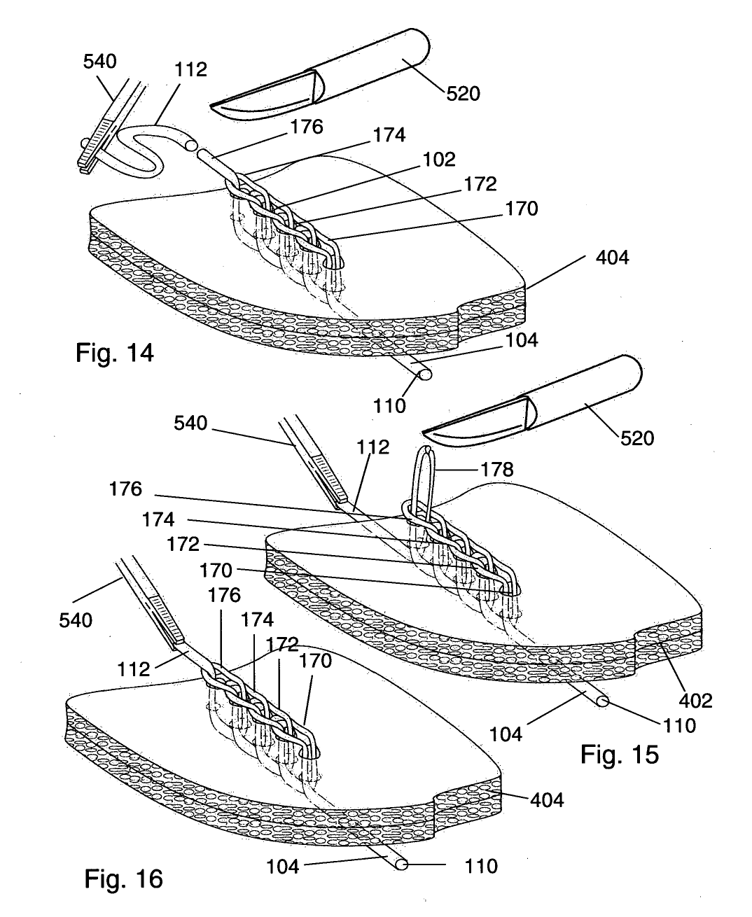

As seen in FIG. 14, following formation of loops 170, 172, 174 and 176 (or more loops as necessary to secure plication 404), a forceps 540 pulls distal suture end 112 through plication 404 and endoscopically introduced tool 520 cuts distal suture 112. Forceps 540 is then used to pull distal suture 112 to tighten loops 170, 172 and 174, thereby securing plication 404.

Alternatively, as seen in FIGS. 15 and 16, a loop 178 is cut, and forceps 540 pulls distal suture 112 to secure plication 404.

In still a further embodiment, not shown, distal suture 112 and loop 176 are joined together with a securing piece, for example a staple, and distal suture 112 is cut beyond the securing piece.

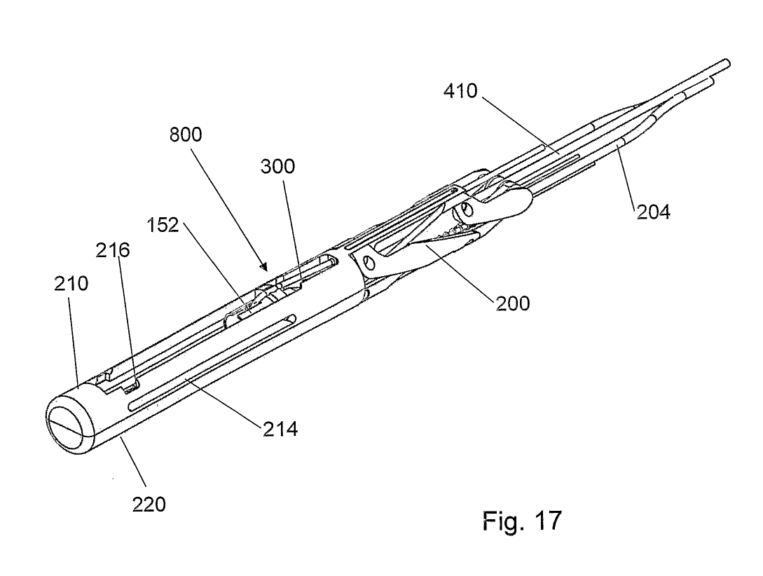

FIG. 17 is an isometric view of suturing assembly 800 in a compact configuration which includes two plates 210 and 220 and a plate opening mechanism 200 that enables the opening or closure of plates 210 and 220 in a substantially parallel configuration.

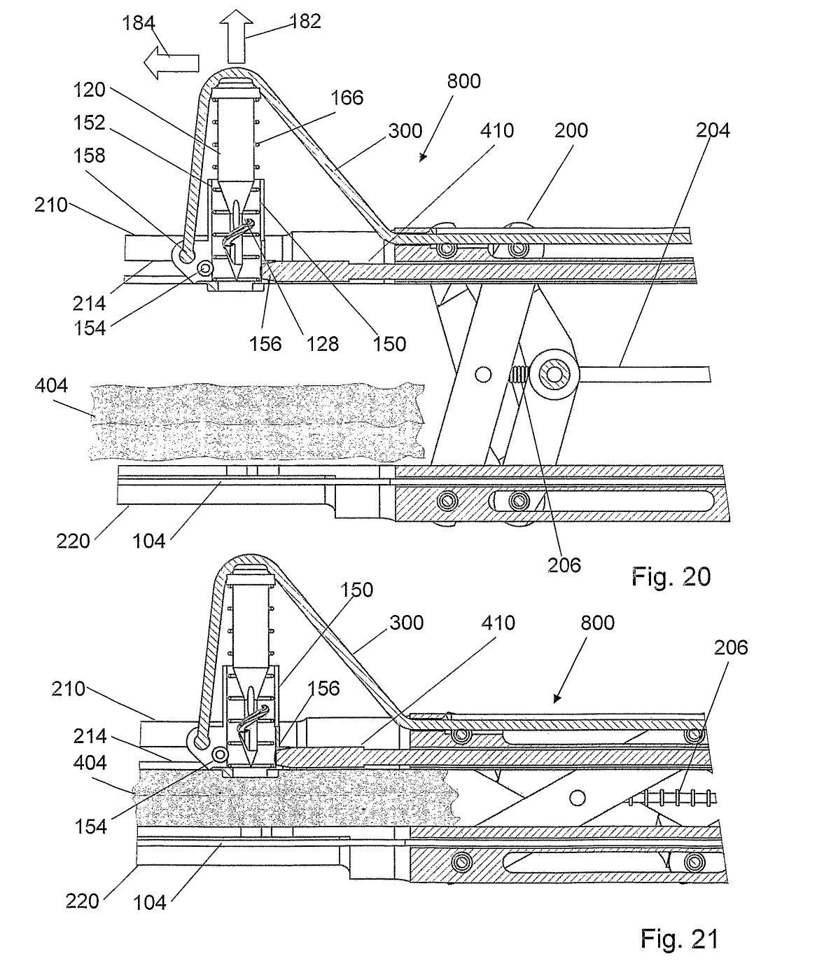

Plates 210 and 220 are separated in FIG. 19 to enable the transition of suture needle 120 from parallel orientation to perpendicular orientation and then to allow introduction of plication 404, as shown in FIG. 20.

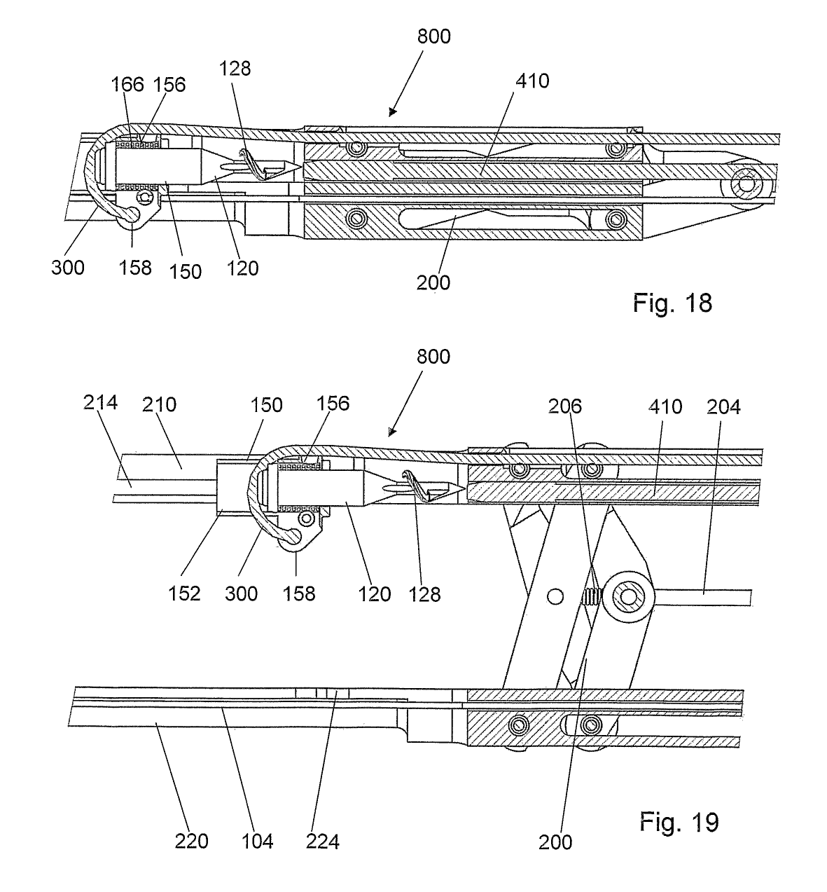

As seen in FIGS. 18-20, a spring 206 causes axial movement of a plate control rod 204 that presses plates 210 and 220 to clamp plicate tissue 404 to provide the above-noted exsanguination. In alternative embodiments, a threaded shaft on plate control rod 204 controls both opening and closing of plates 210 and 220 through rotation.

Pulling a tension cable 300 (FIG. 19) creates tension that causes an offset 158 to rotate, causing suture needle 120 to move from a position that is parallel to plate 210 to a position that is substantially perpendicular to plate 210 (FIG. 20).

With suture needle 120 in the perpendicular position, an engagement bar 410, contained within a suture needle guide 152, is pressed in forward direction 184 by the operator to engage an engagement receptacle 156 so that suture needle 120 becomes fixed in position.

Operation of engagement bar 410 by the operator may be through, inter alia, a nut and thread, or a ratchet mechanism (not shown). Additionally, suturing assembly optionally includes external markers on engagement bar 410 to apprise the operator that suture needle 120 is in the proper position.

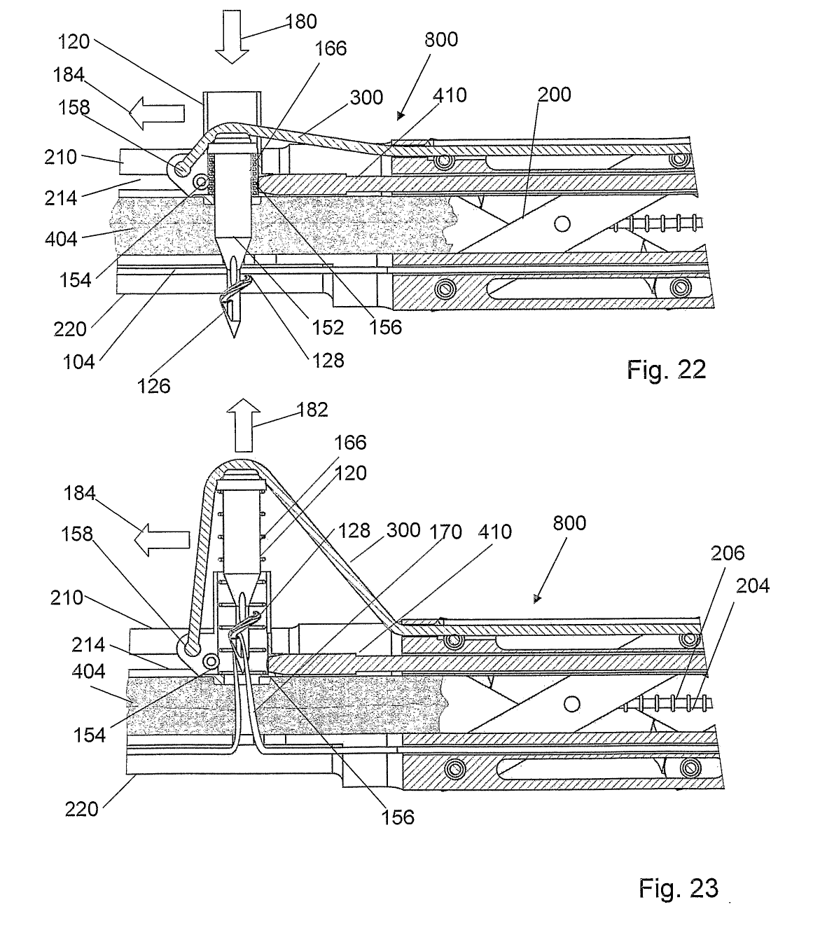

As shown in FIGS. 20 and 21, by releasing tension on tension cable 300, a spring 166 causes suture needle 120 to move in upward direction 182, perpendicular to a plate 214, alternatively referred to as slotted plate 214 or rails 214. As shown in FIG. 22, when the operator creates tension on tension cable 300, suture needle 120 moves in downward direction 180. Further tension on tension cable 300 causes suture needle 120 to move further in direction 180 and pass through plication 404.

In some embodiments, tension cable 300 is connected to a release latch (not shown) that prevents suture needle 120 from moving downward unless an optimal exsanguination pressure is measured in plication 404. Exsanguination pressure in plication 404 may be measured, for example by a load cell (not shown), located in either of plates 210 and 220.

Alternatively, tension cable 300 is prevented from moving suture needle 120 unless plication 404 reaches a preset thickness. The preset thickness of plication 404 may be measured by a distance gauge operatively associated with opening mechanism 200 and/or plates 210 and 220.

As seen in FIG. 23, the forward distance in direction 184 of suture needle 120 is optionally controlled by the operator, for example by rotating advancement rod 204.

In some embodiments, the operator may observe forward movement in direction 184 of suture needle 120 using an endoscope (not shown) and vary the distance of each subsequent loop 170 as desired. Variation in the size of loops 170 may be desirable, for example, in plicating an area around an ulceration wherein the operator wishes to avoid suturing through diseased tissue.

Additionally or alternatively, the distance of each forward movement in direction 184 of suture needle 120 can be preset by the operator with a trigger mechanism (not shown). In some embodiments, the trigger mechanism may optionally be operationally associated with tension cable 300 so that movement of suture needle 120 occurs automatically following completion of each loop 170.

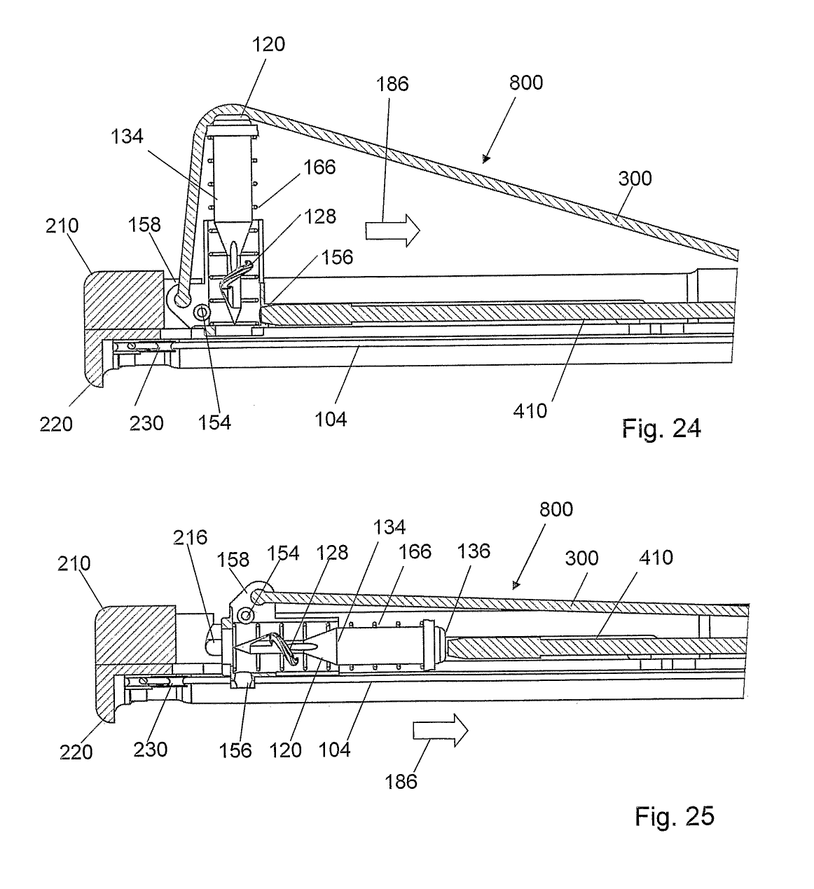

As seen in FIG. 24, following completion of the plication procedure, the operator pulls engagement bar 410 in a rearward direction out of engagement receptacle 156. The operator then pulls tension cable 300 in a rearward direction 186 (FIG. 25) so that suture needle 120 rotates to a compact position.

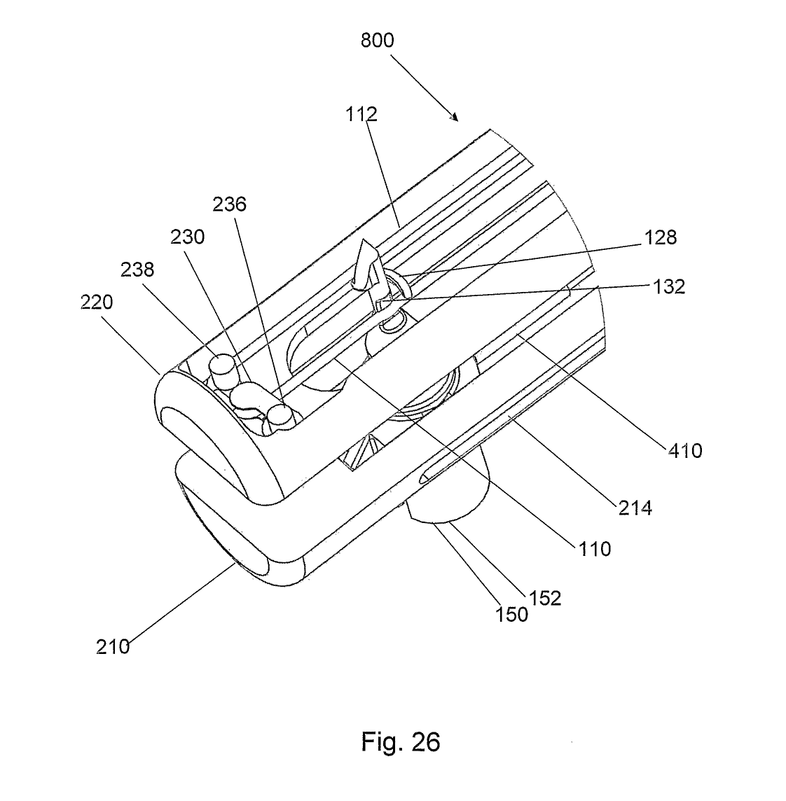

FIG. 26 shows details of the lower surface of suturing assembly 800 in which suture 110 is positioned in groove 132.

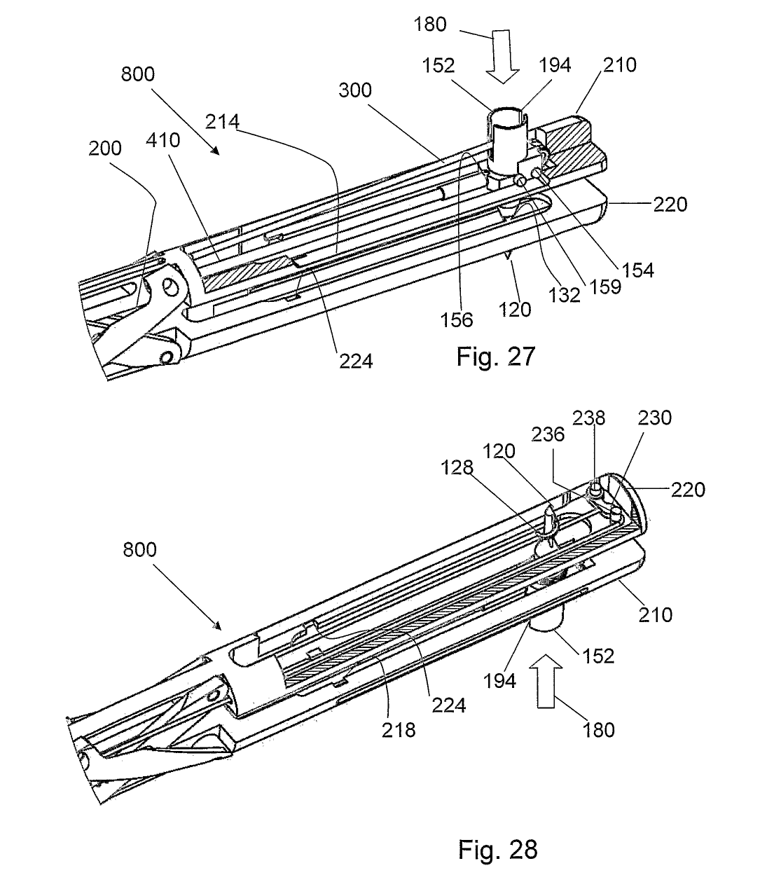

FIGS. 27 and 28 are upper and lower perspectives, respectively, of portions of suturing assembly 800 wherein plates 210 and 220 have been partially removed to better view suture needle 120.

Suture needle guide 152 guides suture needle 120 during movement with respect to the tissue.

Suture needle guide 152 optionally includes cable passage grooves 194 through which cable 300 passes as suture needle 120 passes downward. In embodiments, suture needle 120 further includes a pivot axis 159 around which suture needle 120 pivots between the above-noted compact and perpendicular positions.

A limiting button 154 interacts with plate 214 to prevent suture needle 120 from passing beyond the perpendicular position during plication; such that suture needle 120 slides within suture needle guide 152 and limiting buttons 154 slide within a perpendicular bore within suture needle guide 152.

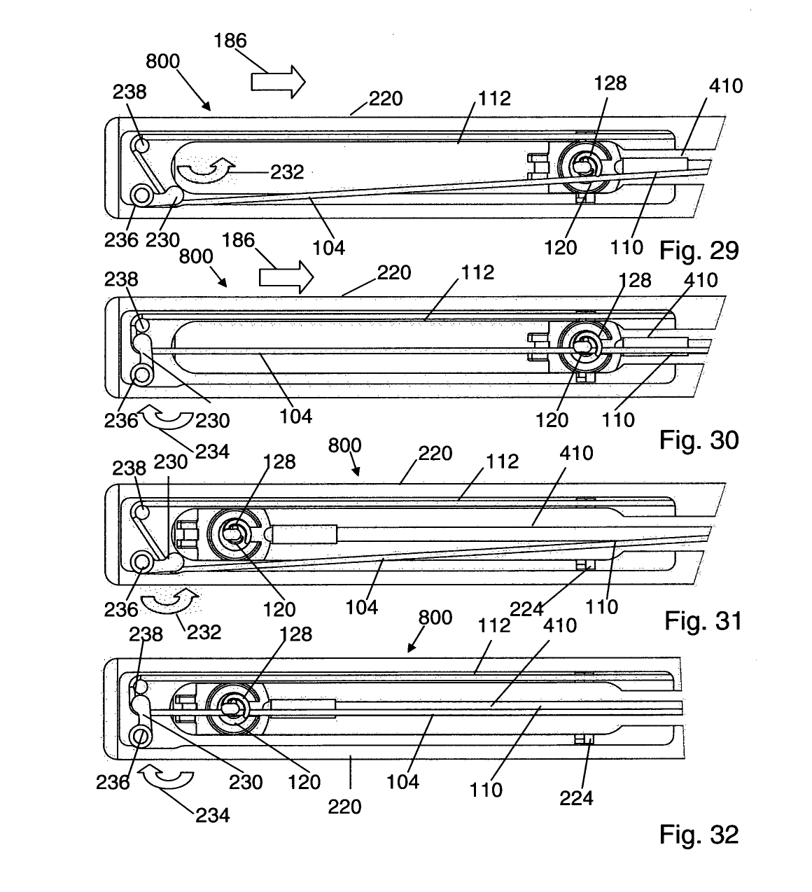

FIGS. 29-32 are views of the lower surface of suturing assembly 800.

FIGS. 29-30 demonstrate that pulling distal suture 112 causes rotation of lever 230 as noted above, thereby moving suture 110 to the center of plate 220 to latch against suture catch 128 in preparation for being pulled through the tissue.

FIG. 32 shows suture needle 120 following creation of a plication and FIG. 31 shows lever 230 rotating in reverse direction 234 to disengage suture 104 from suture catch 128.

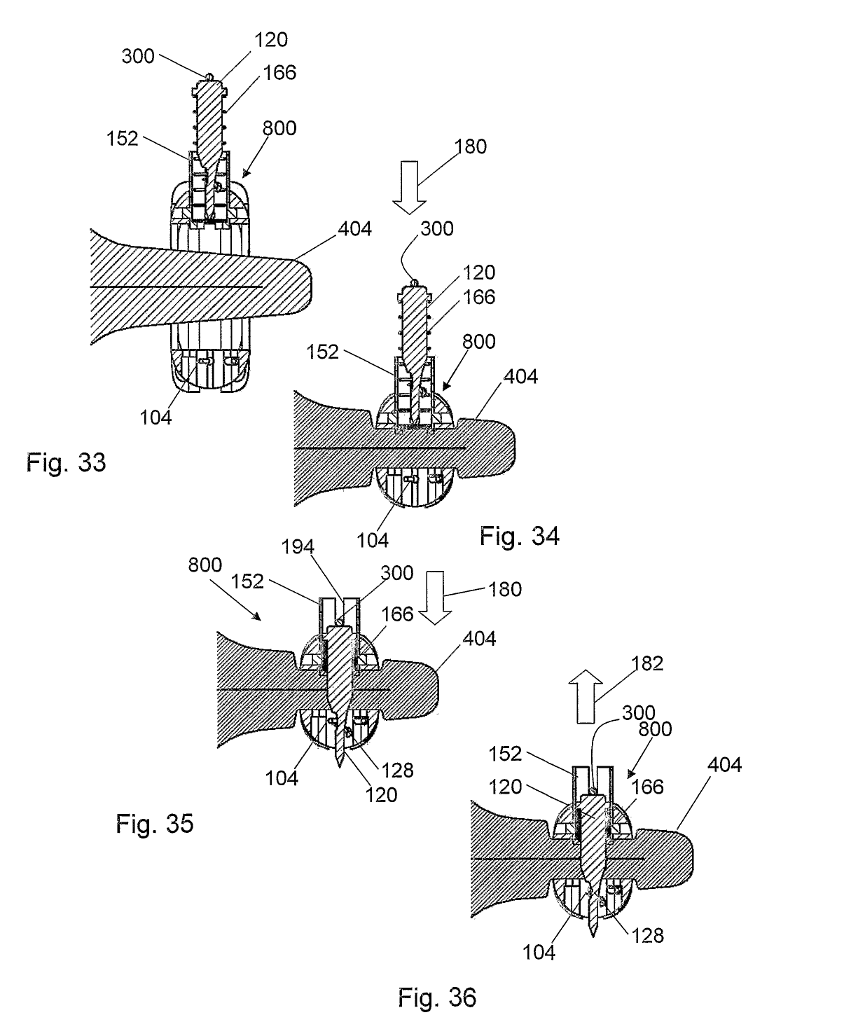

FIGS. 33 to 36 show front-end cross-section views of suturing assembly 800 and plication 404.

During plication, cable 300 passes through cable passage grooves 194 while spring 166 compresses, and suture needle 120 moves in downward direction 180; and suture catch 128 catches a portion of suture 104 (FIG. 35).

FIG. 36 shows suture needle 120 moving in upward direction 182 through plication 404.

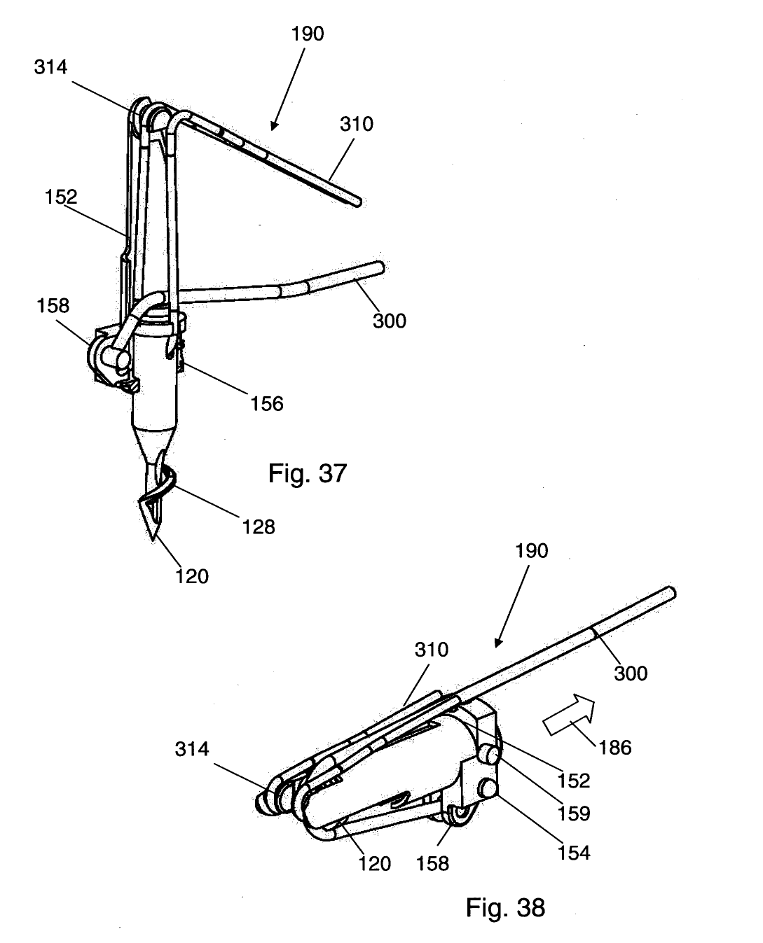

FIGS. 37-38 show a suture needle embodiment 190 comprising elongate suture needle guide 152 that includes guides 314 over which a pulling cable 310 passes. Pulling cable 310 is attached to an external spring mechanism (not shown) and replaces spring 166 (FIG. 34).

FIG. 38 shows suture needle embodiment 190 in its compact configuration following deployment in which pulling cable 310 and cable 300 have been pulled in a direction 186 to cause suture needle embodiment 190 to fold against plate 210.

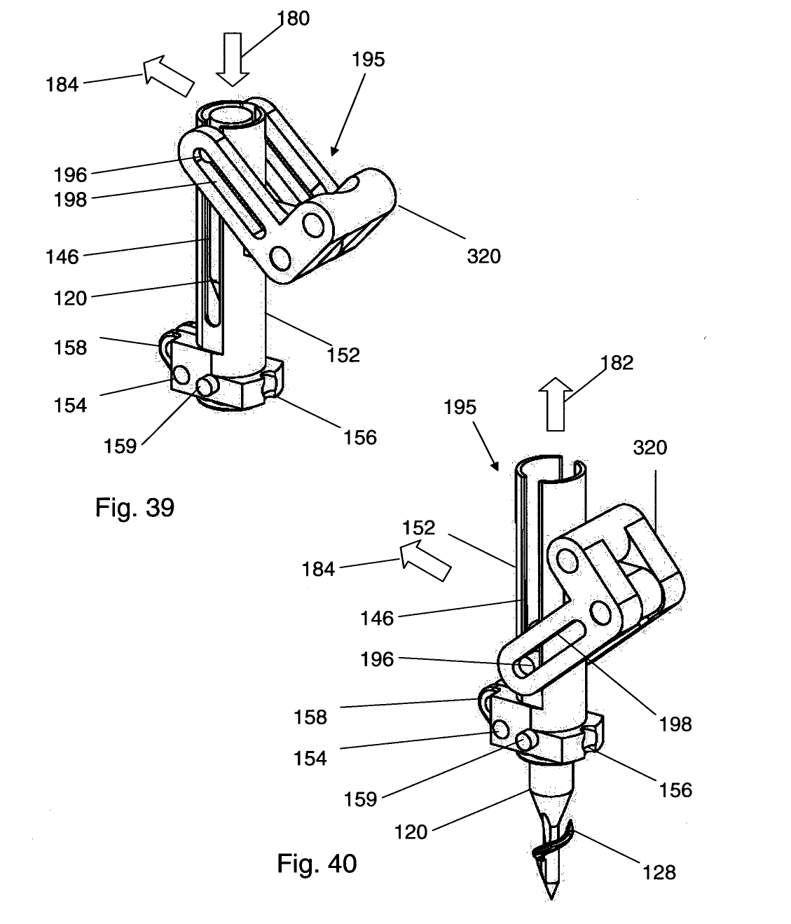

FIGS. 39-40 show a mechanical suture needle assembly 195 in which a bracket joint 320 interacts with suture needle 120 to cause suture needle 120 to move in downward and upward movement 180 and 182 respectively.

Slide posts 196 project from the sides of suture needle 120 and pass through needle housing side grooves 146 into guide apertures 198 which guide suture needle 120 during downward and upward movement 180 and 182, respectively.

In embodiments, at least two procedures can govern the distance that suture needle 120 moves in forward direction 184:

In a first procedure each movement in direction 184 is controlled by the operator. In a second procedure, all movement in direction 184 is preset into mechanical needle assembly 195 and governed, for example, by a spring release upon reaching a preset distance guided by a notch.

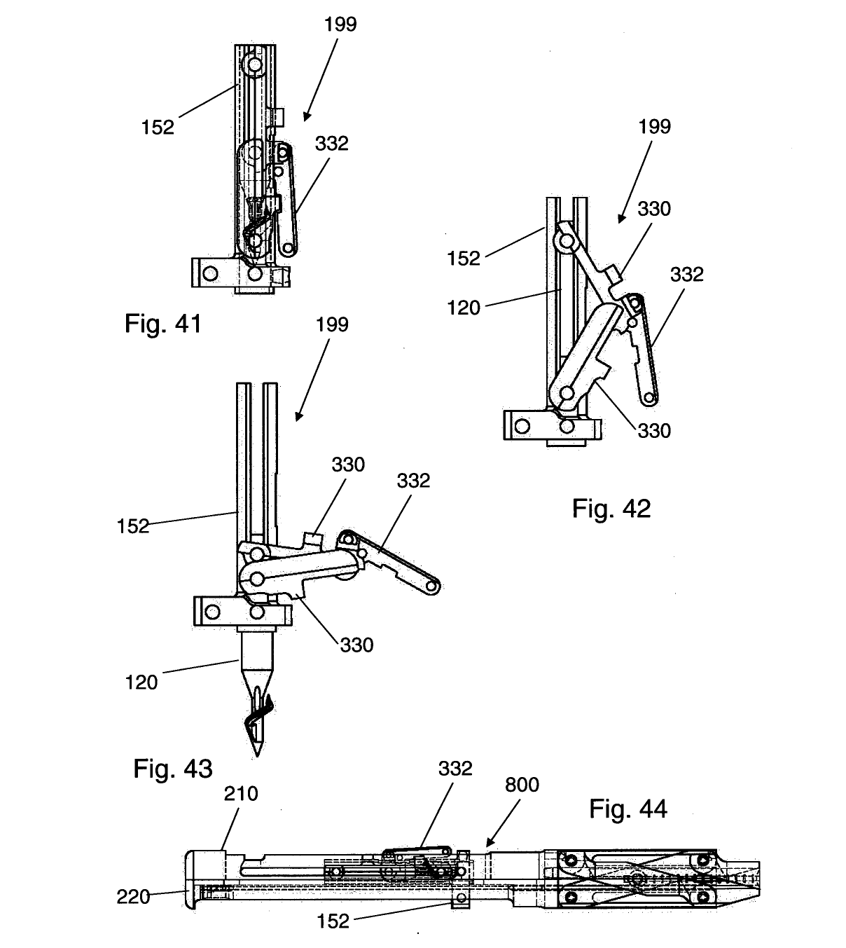

FIGS. 41-44 show alternative embodiments for suture needle activation, in which a mechanical suture needle assembly 199 includes an actuation handle 332 connected to levers 330 which cause suture needle 120 to move upward and downward in needle guide 152, in directions 180 and 182 respectively.

### Lower Surface Chain Stitch

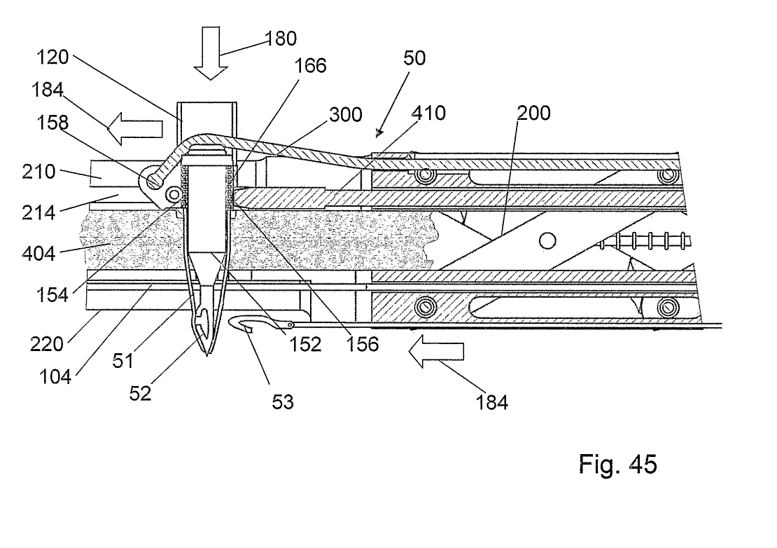

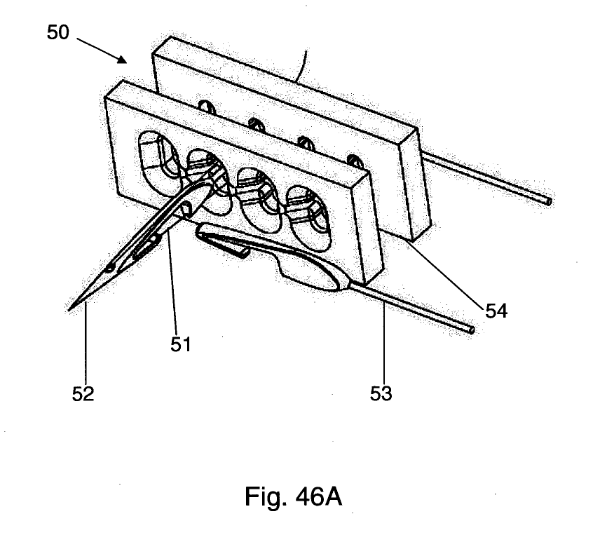

FIG. 45 shows a lower surface chain stitch assembly 50 in which chain stitches are created with a suture needle 51, a suture catch 52, and a hook 53 that move in a synchronized manner.

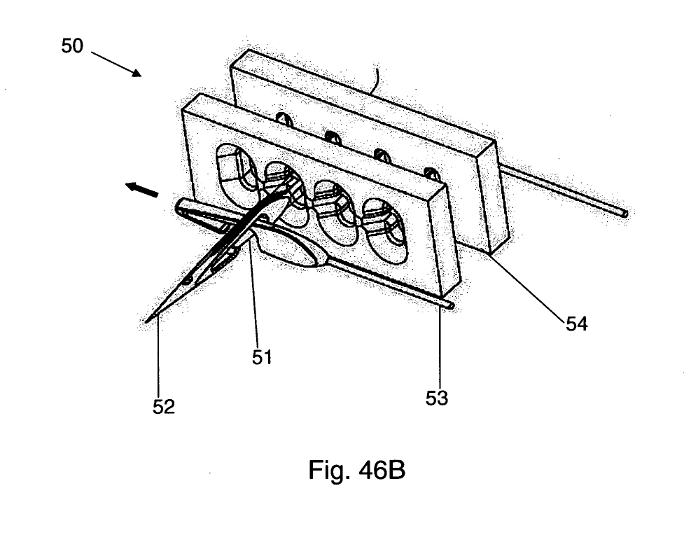

FIGS. 46A-54A show chain stitch assembly suturing a length of chain stitches.

As seen in FIG. 46A, suture needle 51 penetrates through the tissue, and hook 53 moves linearly and perpendicular to suture needle 51 (FIG. 46B) to pass through the gap between needle 52 and suture needle 51.

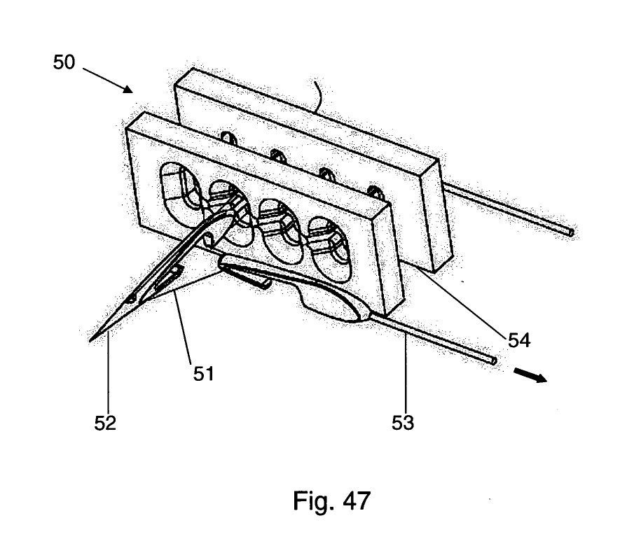

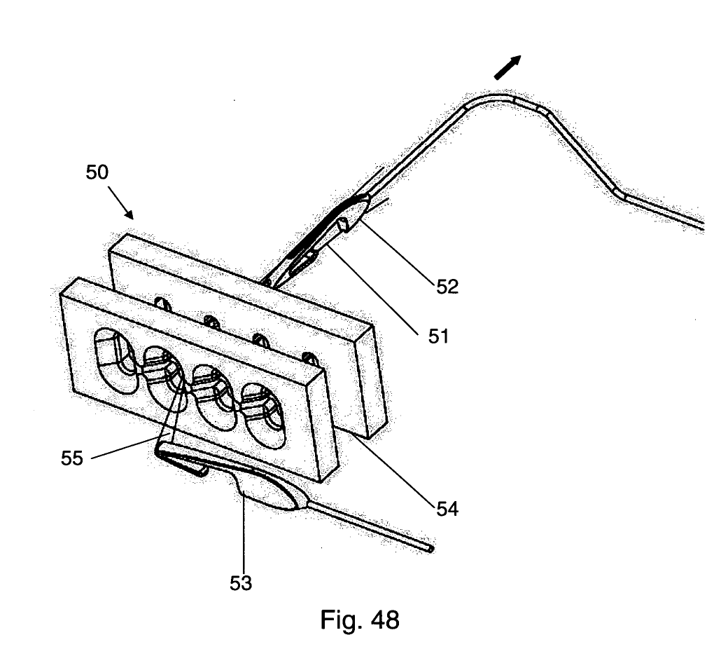

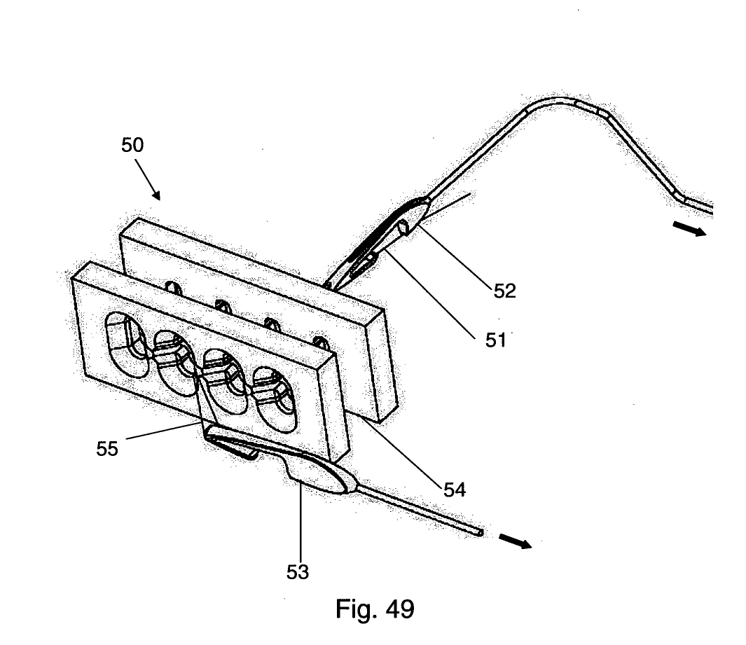

As seen in FIG. 47, when pulled, on its way back, hook 53 traps the needle's suture 52 and creates a loop 55 (FIGS. 48-49) after suture needle 51 retracts to its original location.

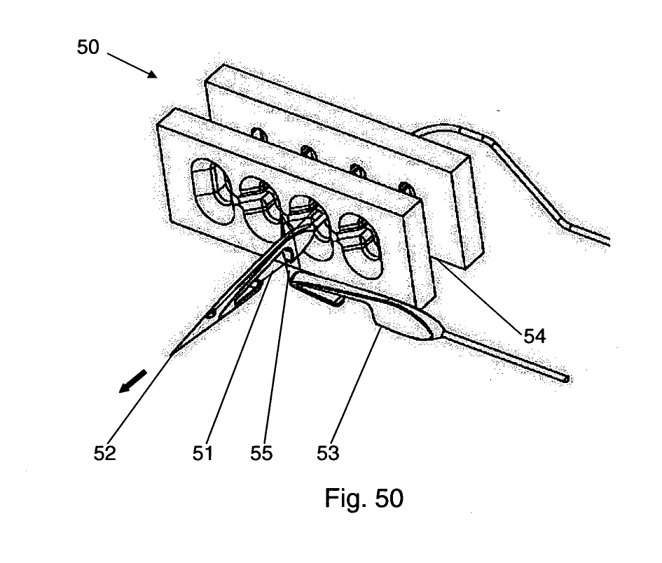

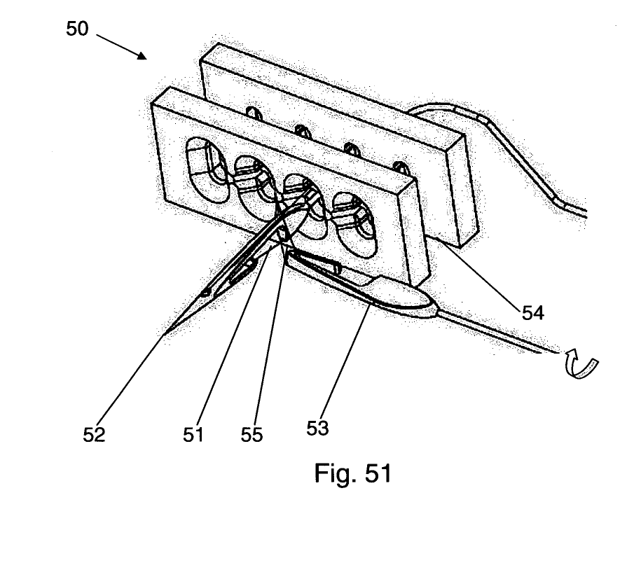

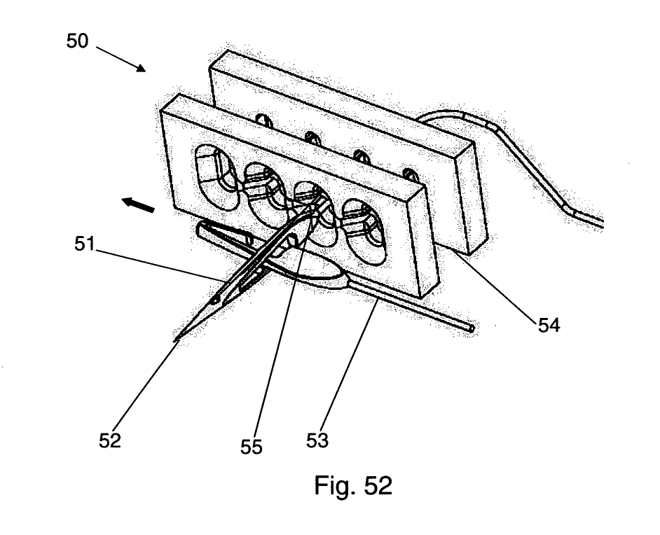

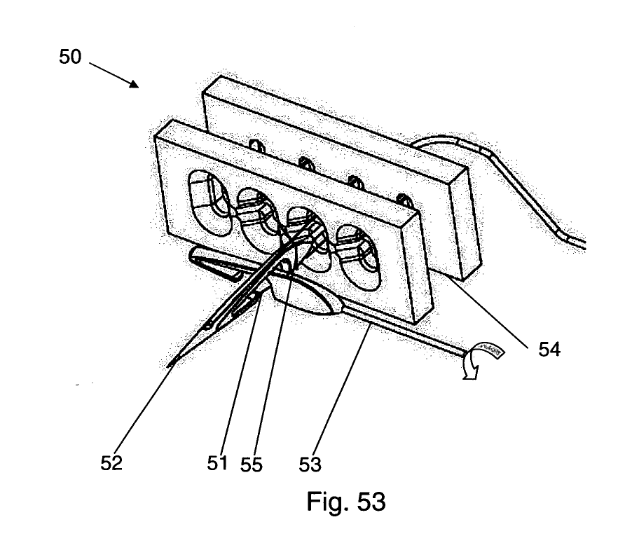

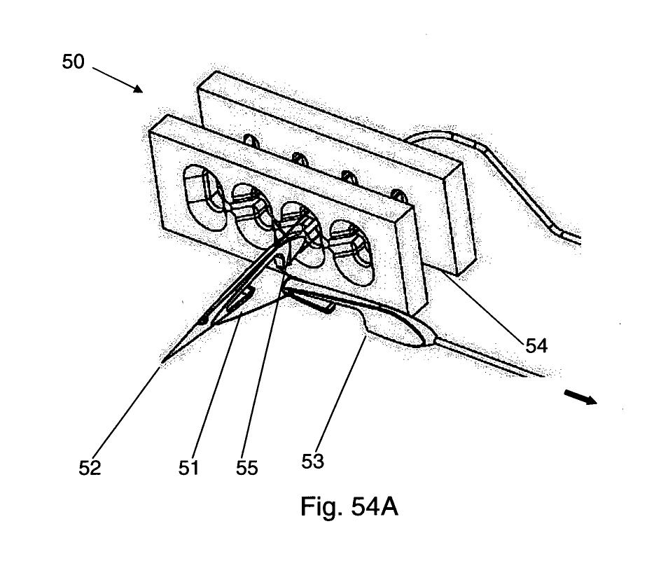

As seen in FIG. 50, suture needle 51 penetrates the tissue and moves through loop 55 held by hook 53. Hook 53 turns on its axis a quarter of a turn in order to enable the release of loop 55. As seen in FIG. 51, hook 53 rotates a quarter turn back to its original position which allows release of loop 55 (FIG. 52). Hook 53 moves forward again along the side of suture needle 51, rotates (FIG. 53) and grasps the needle's suture 52 to form another loop 55 (FIG. 54A).

FIGS. 54B-54D show a compression unit 60 for incorporating lower surface chain stitch 50 and embodiments thereof, into an endoscopic tool. Compression unit 60 ensures that lower surface chain stitch 50 inserts through the esophagus with a minimal cross sectional footprint, after which compression unit 60 is expanded by the operator. Insertion is performed in minimal volume while expansion enables the achievement of an easier approach to the inner stomach walls.

FIG. 54B shows the unit in its position as inserted, FIG. 54C shows it in its expanded position, and FIG. 54D shows it in its final position, with the pressure plates pressing the side by side plications, ready for suturing.

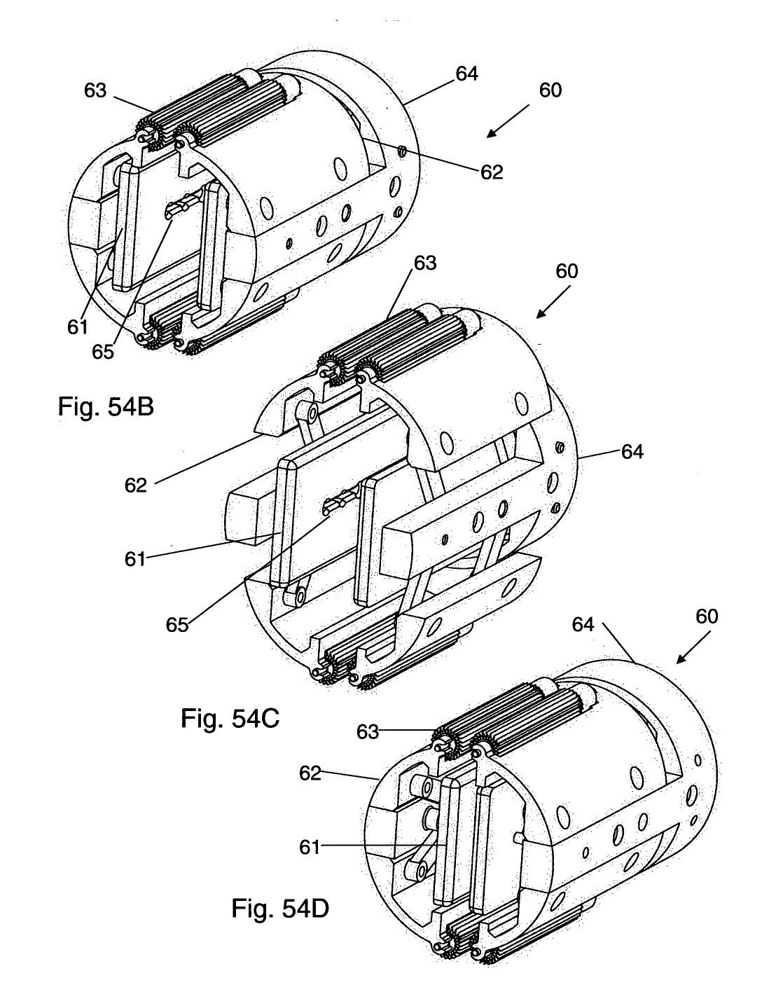

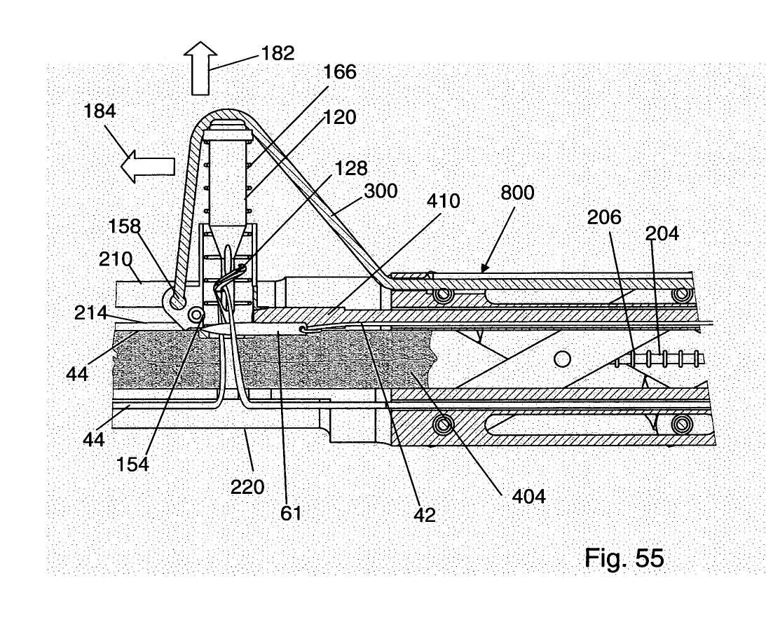

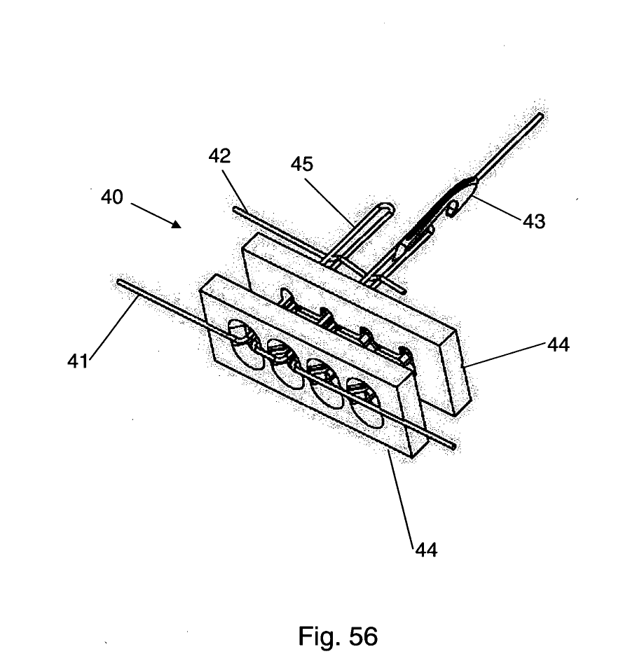

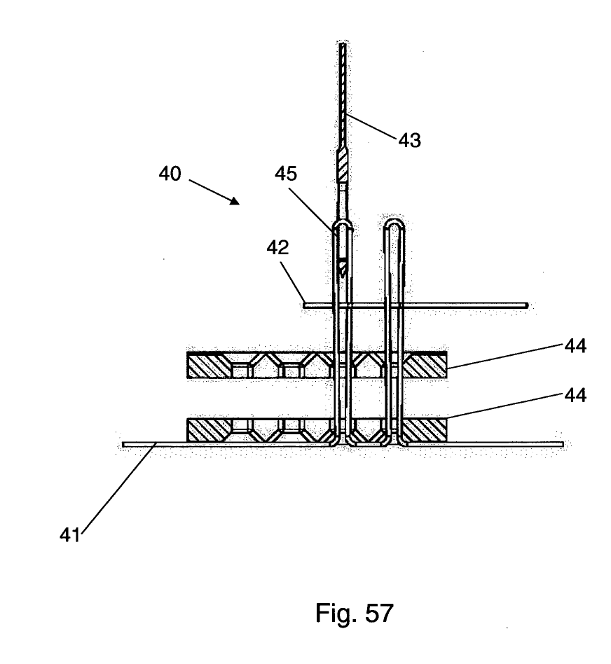

FIG. 55 shows an upper surface lock stitch assembly 800, according to embodiments of the invention.
FIG. 56 shows an isometric view of double wire suturing plates 44 with a suture needle 43, and FIG. 57 shows a cross-section view of the embodiment in FIG. 56.

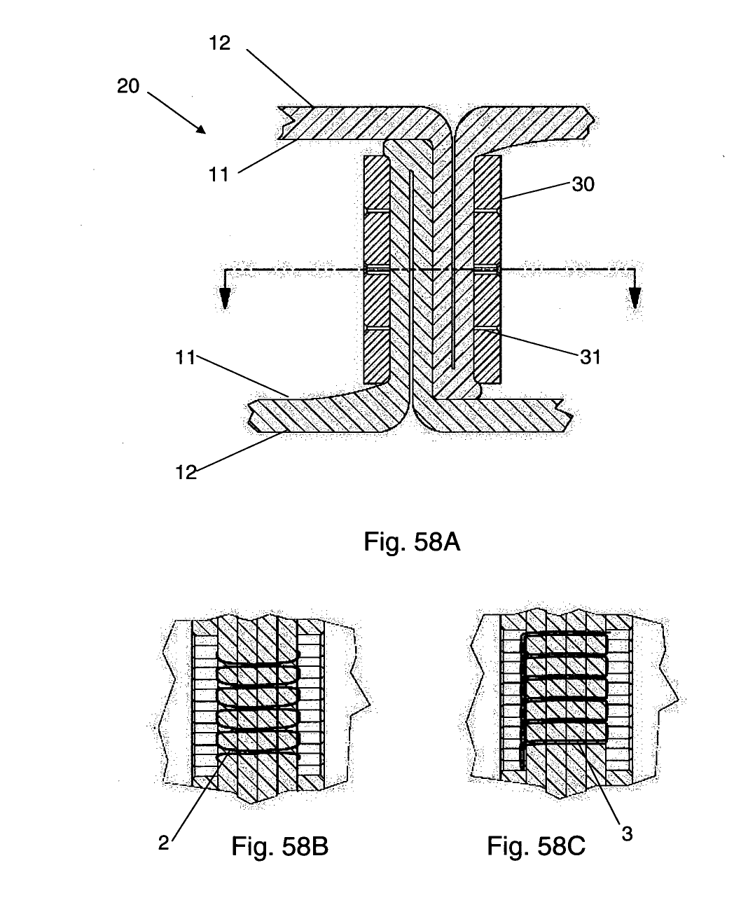

FIG. 58A shows dual plications 12 in which the gastric anterior and posterior walls are trapped by pressure plates 30 in a way that enables suturing under mechanical pressure. FIG. 58B shows a two wire stitch 2, while FIG. 58C shows a single wire stitch 3.

FIG. 59 is an isometric view of the assembly shown in FIG. 58A in which each pressure plate 30 includes a perforation pattern comprising rows of holes 32. Each of holes 32 is connected by a slit 31 through the whole of its depth, so that the sutured thread can be pulled tight through the slit onto the tissue, with the pressure plates still in position, maintaining a minimal area of unpressed plication tissue. Holes 32 guide the penetrating element at a preset pitch between the holes.

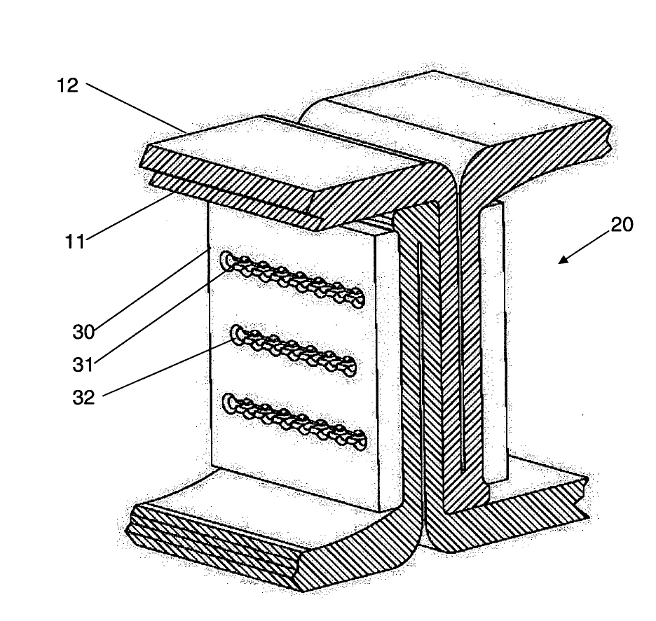

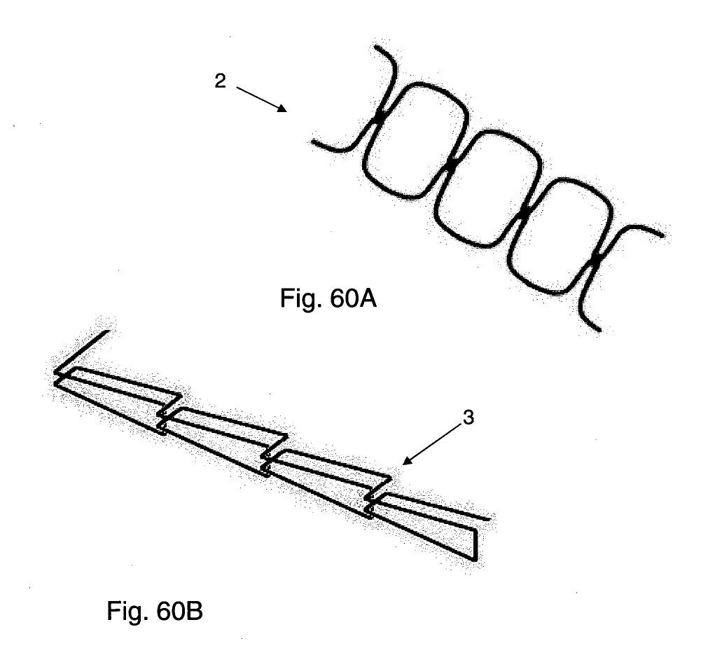

FIG. 60A shows a length of lock stitches 2 formed threads with the assembly shown in FIG. 55. FIG. 60B shows a length of chain stitches formed using a single suture thread using the assembly shown in FIG. 45.

FIG. 61 shows a prior art view of a plication 10 created with sutures without pressure plates in which the outer surfaces comprise mucosa 11 and the inner surfaces comprise the serosa.

Double row suturing assembly

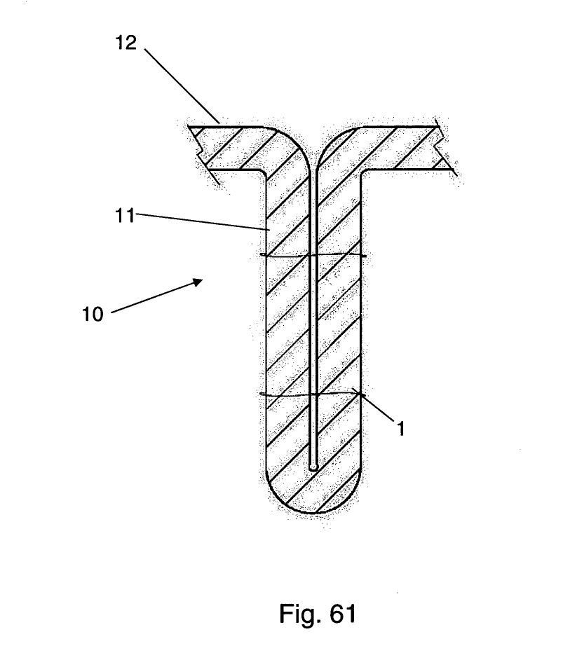

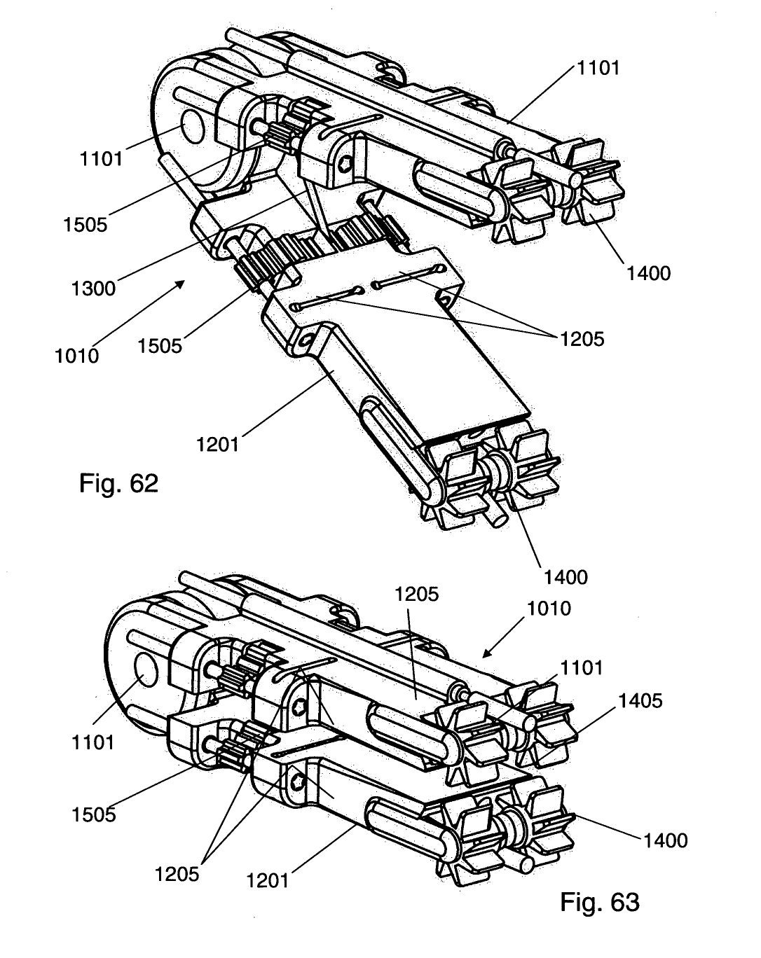

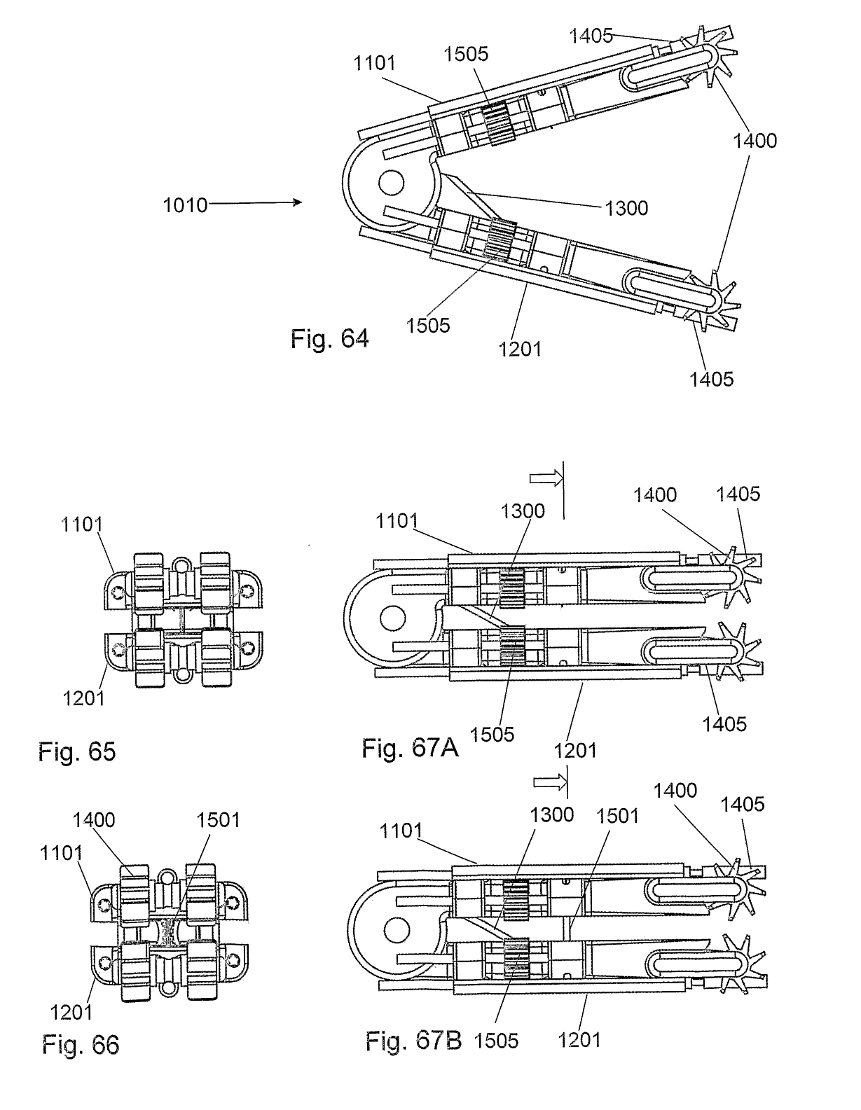

An open configuration of a double row suturing assembly 1010 is shown in FIG. 62 in which the tissue can be positioned between pressure plates 1101 and 1201. A closed configuration, alternatively referred to as a compact or compressed configuration, of double row suturing assembly 1010 is shown in FIG. 63, in which the tissue can be sutured and cut by double row suturing assembly 1010. FIG. 64 shows a side view of double row suturing assembly 1010 in the open configuration; FIGS. 65-66 show frontal views of double row suturing assembly 1010 in the closed position, and FIGS. 67A-67B show side views of double row suturing assembly 1010.

In embodiments, double row suturing assembly 1010 includes a cutting blade 1300 (FIGS. 67A-67B) that optionally remains in a relatively fixed position during suturing and cuts the tissue as double row suturing assembly 1010 is moved forward during suturing. In embodiments, pressure plates 1100 and 1200 extend from a rotatable connection 1101 and the operator is able to control the pressure generated by double row suturing assembly 1010 by rotating pressure plates 1100 and 1200 around rotation connection 1101. The rotation connection actuator is not shown but optionally comprises any one of a large variety of actuators including cables and other mechanisms described above.

Each pressure plate 1100 and 1200 includes assembly movement systems 1405 comprising cogwheels 1400 that enable assembly 1010 to move linearly forward along the tissue as the cogs on cogwheels 1400 grasp the stomach tissue while cogwheels 1400 rotate.

Movement systems may optionally include, inter alia; shafts, cams, and/or cables that enable external control of the operation of assembly movement system 1405.

Each pressure plate 1100 and 1200 additionally includes suture needle positioning wheels 1505 that rotate needles 1501 counterclockwise through the tissue and clockwise out of the tissue. While needles 1501 are shown as being curved, alternative embodiments using straight needles as in embodiments presented above, may be configured into double row suturing assembly 1010.

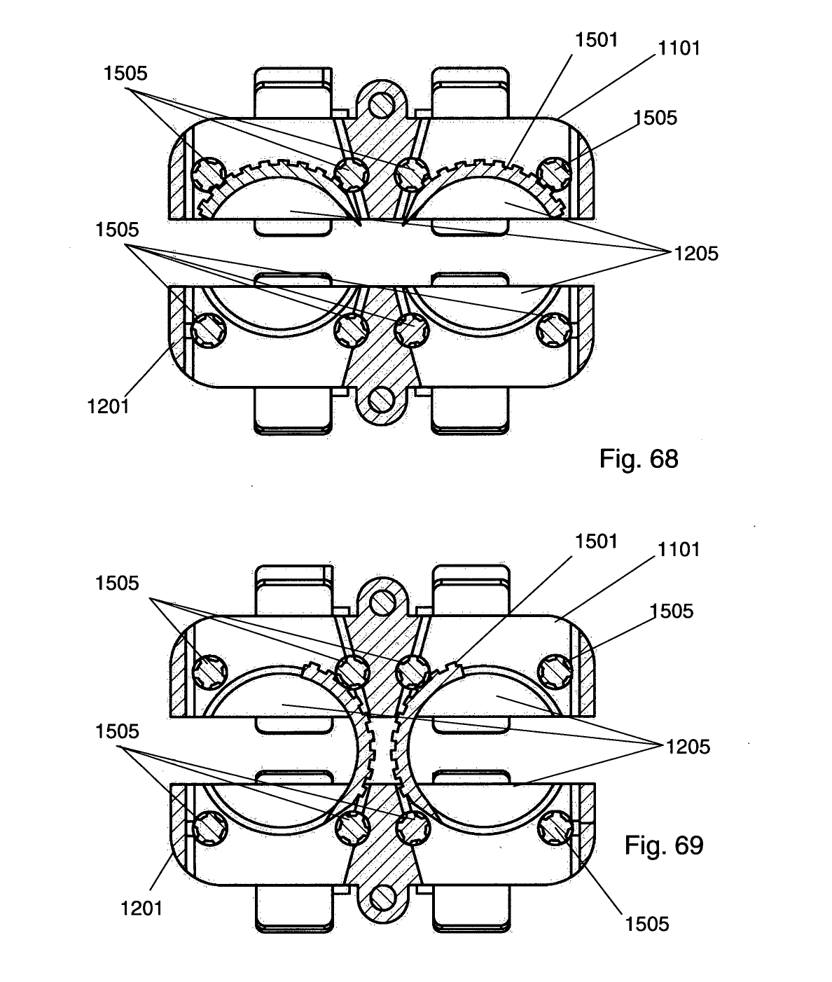

FIGS. 68-69 are cross-section views following the cross-section line shown in FIG. 67A and show suture needle positioning wheels 1505 interfacing with needles 1501. Suture needle positioning wheels 1505 are optionally connected to an actuator that extends out of the stomach and body which simultaneously actuates the movement of needles 1501.

A slit 1205 enables the extraction of the suturing wire from double row suturing assembly 1010 following completion of stitching of dual rows of stitches.

Gastric partitioning using double row suturing assembly 1010 is optionally performed using an endoscopic procedure in which double row suturing assembly 1010 is inserted via the esophageal channel into the stomach. Prior to inserting suturing assembly 1010 or plication instruments, food and fluids are optionally drained from the stomach. Additionally, prior to suturing, plication between two gastric walls is optionally created using endoscopic instruments known in the art, as explained below.

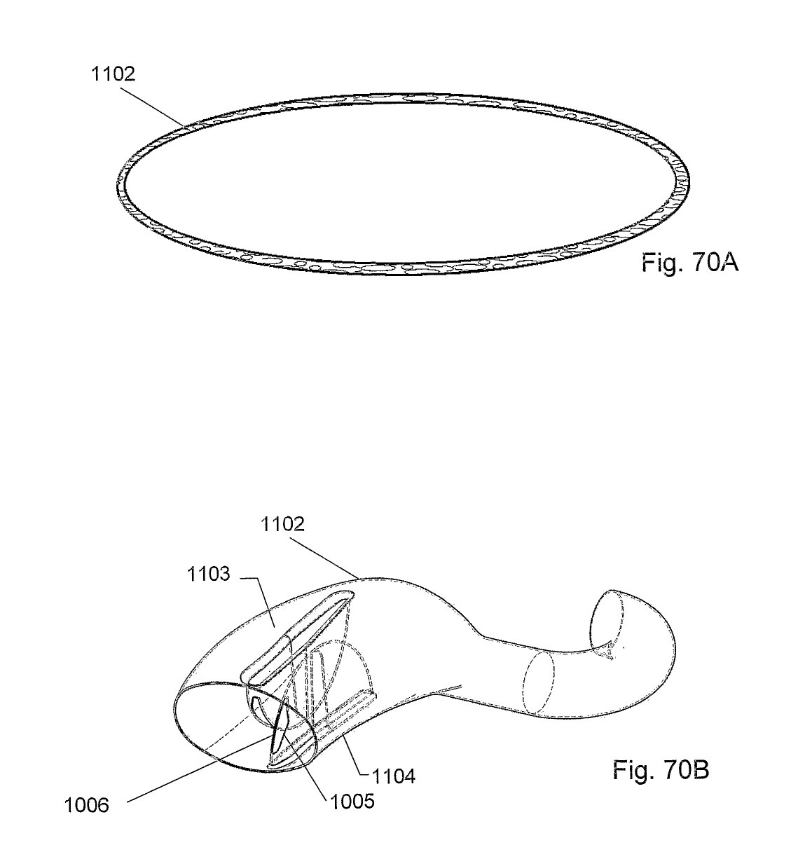

FIG. 70A shows a typical cross-section of the stomach interior. FIG. 70B shows planned partition. FIG. 70C shows a side view of the stomach following suturing along the planned partition shown in FIG. 70B. The posterior and anterior gastric walls have been sutured using two rows of stitches, and a cutting blade has cut between the suture rows to create the partition.

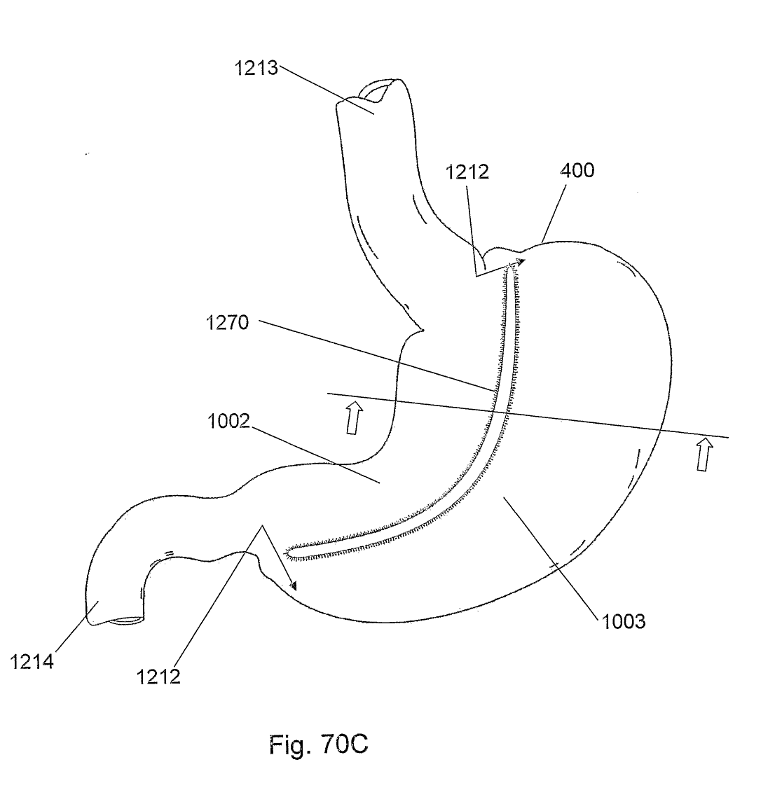

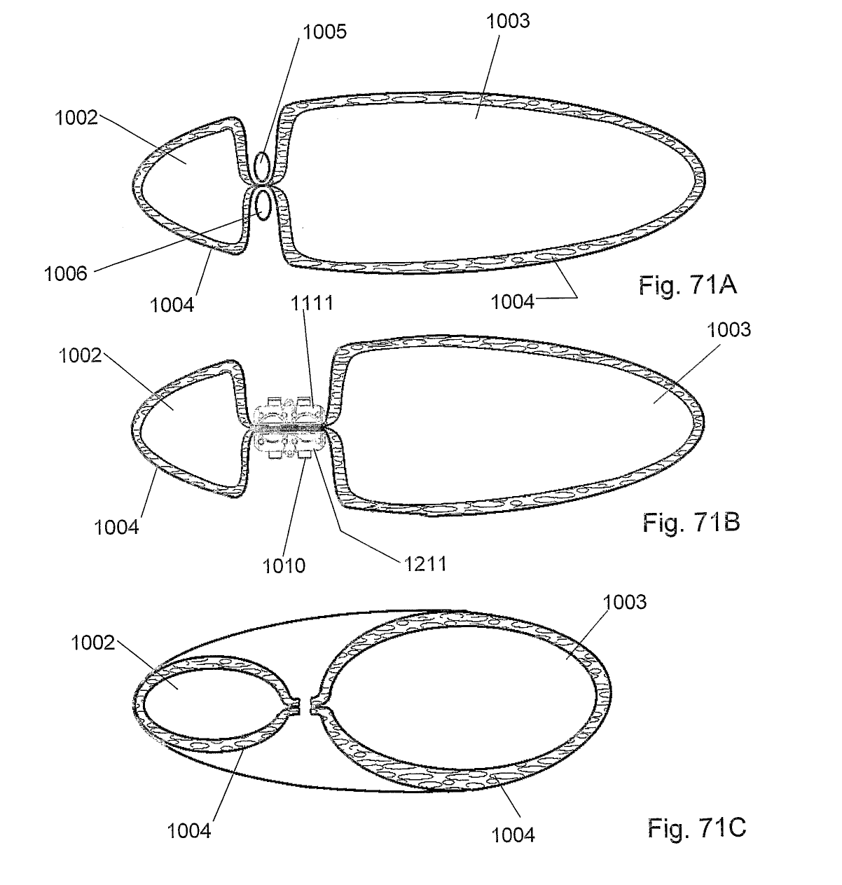

FIG. 71A shows the compression of the stomach walls in preparation for suturing. Suction tubes 1005 and 1006 have been passed through a portal in the abdominal wall under endoscopic observation.

Upon proper positioning along the serous membrane on the outer gastric surface, herein serosa 1004, the operator fixes the position of suction tubes 1005 and 1006 by activating a suction pump that temporarily adheres suction tubes 1005 and 1006 to the serosa. The operator then activates a compression actuator that presses the gastric walls together as shown. Optionally, the operator endoscopically introduces a second set of suction tubes (not shown) to create a sufficiently wide compressed gastric area for introduction of double row suturing assembly 1010 through an abdominal portal.

Suction tubes 1005 and 1006 (FIG. 71A) allow the positioning and subsequent movement of suturing assembly 1010 within the created troughs in the gastric tissue with substantially minimal risk of damage to neighboring organs.

In alternative embodiments, other gastric compression instrumentation may be substituted for suction tubes 1005 and 1006; the many compression instruments being well-known to those familiar with the art.

FIG. 71B shows the location of double row suturing assembly 1010 which are positioned over suction tubes 1005 and 1006, the suction action is ceased and suction tubes 1005 and 1006 are removed and compression surfaces 1111 and 1211 of suturing assembly 1010 are clamped into position along the compressed gastric tissue as seen in phantom lines in FIG. 71B.

With compression surfaces 1111 and 1211 clamped onto the gastric serosa, the operator activates double row suturing assembly 1010 to begin moving forward while suturing and separating the gastric tissue.

FIG. 71C shows a cross-sectional view showing the partitioned sections, to comprising a main compartment 1002 that is formed, through which consumed food is usually digested. A side compartment 1003 allows continuous passage of gastric fluids that may accumulate therein.

Main compartment 1002 and side compartment 1003 are not fully isolated from each other as additionally seen in FIGS. 70C. Additionally, main compartment 1002 receives food passing from the esophagus directly from a gastric inlet 1213, and sends the food, following processing in the stomach, through a gastric outlet 1214.

as seen in FIG. 70C.

Side compartment 1003 remains connected by narrowed passages 1212 to gastric inlet 1213 and gastric outlet 1214, allowing drainage of fluids that may flow into side compartment 1003.

It may happen that side compartment 1003 will fill with food if the patient continues to eat after compartment 1002 is already full. In such cases, the inventors have discovered that side compartment 1003 acts to provide a sensation of satiety by associated slow passage of the food.

Additional elements that may be included in double row suturing assembly 1010 to aid in the partitioning process include a fiber optic visualization system or assembly navigating system, an endoscope, a catheter allowing easy access to actuators located outside the body.

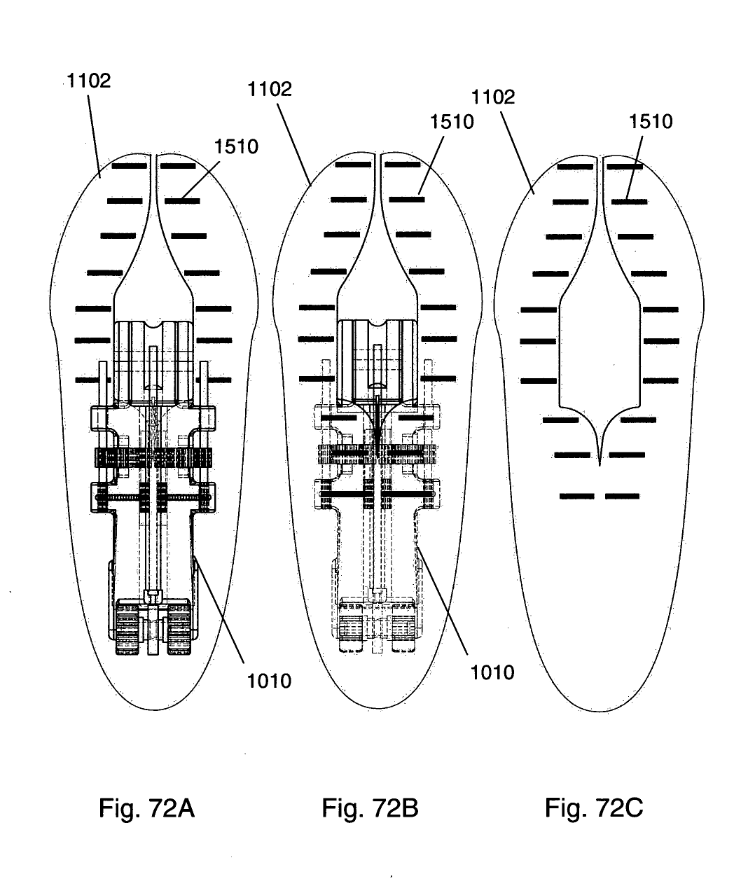

FIGS. 72A-72C show views of a stomach following double row suturing assembly 1010 with sutures 1510 in position. FIG. 72A shows the stomach wall with double row suturing assembly 1010 in a final position with double row suturing assembly 1010 visible.

In FIG. 72B, double row suturing assembly 1010 is shown in phantom to demonstrate a typical space between partitions 1102 with respect to double row suturing assembly 1010.

In FIG. 72C double row suturing assembly 1010 has been removed to show an aerial view of a typical gastric partition.

There are many additional configurations of double row suturing assembly 1010 that may be contemplated; the following being just one such example.

### Straight Needle Double Row Assembly

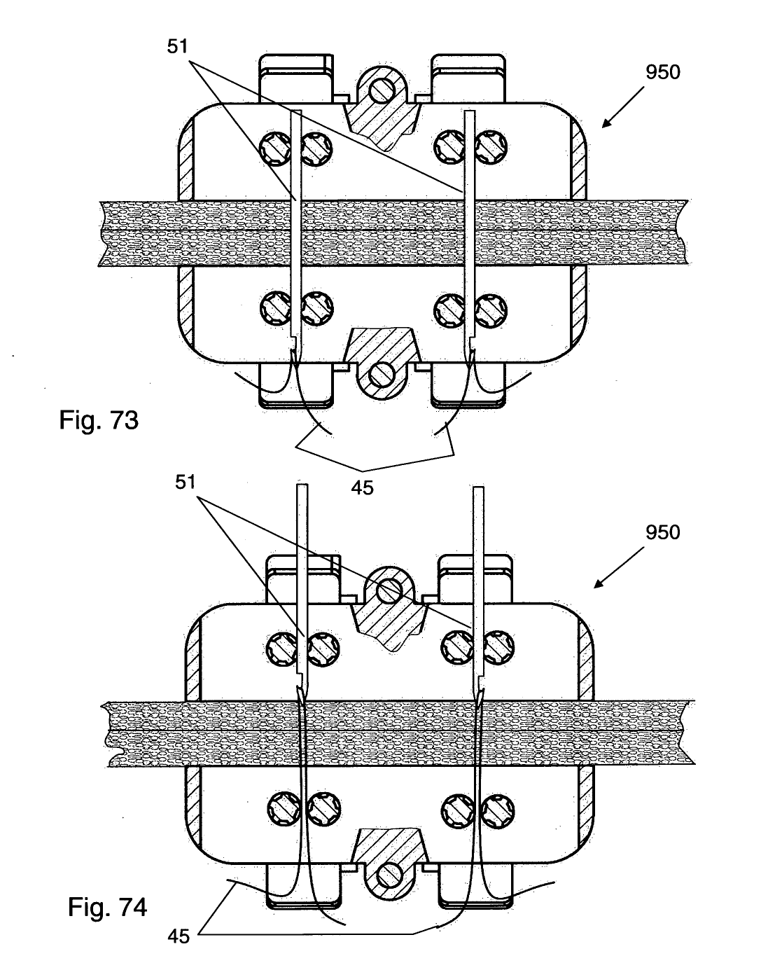

FIGS. 73 and 74 show a double row suture assembly 950 having straight needles with suture 45 passing through in vivo tissue and configured to form a length of upper surface chain stitches. Additionally or alternatively, double row suture assembly 950 may optionally be modified to provide lower surface chain stitches and upper surface lock stitches in similar configurations to suture assembly embodiments explained above.

The stomach wall consists of four major layers. As noted above, the inner layer is the mucosa and the outer layer is the serosa. A first serosa layer is sutured to a second serosa layer. Double row suturing assembly 1010 allows natural tissue healing processes to connect the layers of the serosa; with the sutures supporting the tissue during the healing period.

The inventors have discovered that one possible advantage of leaving side compartment 1003 (FIG. 70C) partially attached to side compartment 1002 can potentially avoids possible changes in blood flow and possibly avoid hormonal balance that may result from complete separation.

It is expected that during the life of a patent maturing from this application many relevant plication devices will be developed and the scope of the term plication device is intended to include all such new technologies a priori.

As used herein the term “about” refers to ±10%

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-56. (canceled)

57. A suturing assembly for suturing in vivo tissue, the suturing assembly comprising:
i) a first suture thread positioned along a first surface of an in vivo tissue, the in vivo tissue having a first surface and a second surface;
ii) at least one suture needle configured to draw the first suture thread through the in vivo tissue; and
iii) a suture catch located on at least one side of the at least one suture needle, the suture catch configured to releasably catch the suture, such that prior to a first passage through the in vivo tissue, the first suture thread extends on a flat plane on either side of the at least one suture needle, and thereafter the at least one suture needle draws the suture through the in vivo tissue during a first passage through the in vivo tissue such that the suture is formed into a first loop on a surface of the in vivo tissue.

58. The suturing assembly according to claim 57, wherein the at least one suture needle is additionally configured to pass through the first loop during a second passage through the in vivo tissue and draw the suture through into second loop through the first loop.

59. The suturing assembly according to claim 58, wherein the at least one suture needle comprises at least two suture needles: at least one first suture needle spaced a distance from at least one second suture needle.

60. The suturing assembly according to claim 59, wherein the at least one suture needle, is at least one of:
a) curved; and
b) straight.

61. The suturing assembly according to claim 60, including one cutting element configured to cut the in vivo tissue between the first loop and the second loop created by the at least one first suture needle spaced a distance from at least one second suture needle.

62. The suturing assembly according to claim 61, wherein the one cutting element cuts the in vivo tissue during at least one of:
i) following formation of the first loop and the second loop; and
ii) during formation of the first loop and the second loop.

63. The suturing assembly according to claim 59, including one elongate plate having a surface configured for pressing against an in vivo tissue, the one elongate plate having at least one aperture configured to allow passage of the suture needle therethrough.

64. The suturing assembly according to claim 58, wherein the first loop and the second loop are formed proximate to the first surface of the in vivo tissue and the assembly includes, proximate to the first surface, a second suture operatively associated with a shuttle.

65. The suturing assembly according to claim 64, wherein the first loop and the second loop each include an inner boundary serially defining spaces having at least two areas:
i) a first area sufficiently large to allow passage of the shuttle and shuttle suture therethrough; and
ii) a second area sufficiently small to compress a portion of the shuttle suture against the first surface.

66. The suturing assembly according to claim 59, wherein the at least one suture needle is rotatably connected to an elongate plate and configured to assume at least two positions:
i) a first position wherein the at least one suture needle is non-perpendicular to the longitudinal axis of the plate; and
ii) a second position wherein the at least one suture needle is perpendicular to the longitudinal axis of the plate.

67. The suturing assembly according to claim 59, including a cutting element configured to cut the in vivo tissue during at least one of:
i) following formation of the first loop and the second loop; and
ii) during formation of the first loop and the second loop.

68. A lock stitch suturing assembly for suturing in vivo tissue, the lock stitch assembly comprising:
i) a suture thread wherein prior to being caught by a suture catch, the suture thread is configured to be positioned along a second surface of an in vivo tissue having a first surface and a second surface;
ii) at least one suture needle having a suture catch, the at least one suture needle and suture catch configured to:
a) pass through a first surface and the second surface of the in vivo tissue;
b) catch the suture thread on the suture catch; and
c) draw the suture thread through the in vivo tissue thereby forming a loop projecting beyond the first surface of the in vivo tissue; and
iii) a shuttle connected to a shuttle suture and configured to pass through the loop, thereby forming a lock stitch following tightening of the loop around the shuttle suture.

69. A chain stitch suturing assembly for suturing in vivo tissue, the chain stitch suturing assembly comprising:
i) a first suture having a longitudinal flat axis extending from either side of a suture needle, the first suture positioned proximate to and along a first surface of an in vivo tissue;
ii) at least one suture needle having a suture catch, the at least one suture needle configured to:
a) catch the first suture on the suture catch; and
b) pass a first pass through the first surface and a second surface of the vivo tissue; and
iii) a hook operatively associated on the second surface and configured to releasably pull the first suture into a loop that radially outwardly extends along the second surface from the at least one suture needle, the loop configured with an inner boundary of a size that is larger than the outside diameter of the at least one suture needle.

70. A chain stitch suturing assembly for suturing in vivo tissue, the chain stitch suturing assembly comprising:
i) a first suture which, prior to being engaged by a suture catch, the first suture is positioned along a first surface of an in vivo tissue;
ii) at least one suture needle having a suture catch, the at least one suture needle configured to:
a) catch the first suture on the suture catch; and
b) pass a first pass through the first surface and a second surface of the in vivo tissue; and
iii) a hook operatively associated on the second surface and configured to releasably pull the first suture into a loop that radially outwardly extends along the second surface from the at least one suture needle, the loop configured with an inner boundary of a size that is larger than the outside diameter of the at least one suture needle.

71. A suturing assembly for suturing in vivo tissue, the suturing assembly comprising:
i) an elongate plate having at least one aperture configured to allow passage of a first suture needle; the first suture needle rotatably and operatively associated with the elongate plate, the first suture needle additionally being rotatable to at least two positions with respect to the elongate plate:
a) a first position wherein the first suture needle is non-perpendicular to the longitudinal axis of the elongate plate; and
b) a second position wherein the first suture needle is perpendicular to the longitudinal axis of the plate;
ii) a suture catch projecting radially outward from at least one side of at least one suture needle, the suture catch configured to releasably catch a portion of a suture when the at least one suture needle is in the second position and passing a first pass through the in vivo tissue; and
iii) a suture, which prior to being caught by the suture catch is configured to be positioned along a second surface of the in vivo tissue having a first surface and a second surface.

72. The suturing assembly according to claim 71, wherein after the at least one suture needle passes through the in vivo tissue, the suture catch is configured to form the suture into a first loop extending from a first surface of the in vivo tissue.

73. The suturing assembly according to claim 72, wherein the assembly includes, proximate to the first surface, a second suture operatively associated with a shuttle, the shuttle and the second suture are configured to pass through the first loop, thereby forming a lock stitch in the in vivo tissue.

74. A suturing assembly for suturing in vivo tissue, the suturing assembly comprising: one elongate plate having a surface configured for pressing against an in vivo tissue, the one elongate plate having two apertures located a distance from each other, a first aperture and a second aperture, each of the two apertures configured to allow passage therethrough of a suture needle;
a) one first suture needle configured to removably connect to a first suture positioned between a surface of the in vivo tissue and the one elongate plate, the first needle configured to pass multiple times through the first aperture and create a first length of stitches in the in vivo tissue; and
b) one second suture needle, configured to removably connect to a second suture positioned between a surface of the in vivo tissue and the one elongate plate, the second needle configured to pass multiple passes through the second aperture and create a second length of stitches in the in vivo tissue.

75. A suturing assembly for suturing in vivo tissue, the suturing assembly comprising:
i) an elongate plate having at least one aperture configured to allow passage of at least one suture needle;
ii) at least one suture needle configured to pass through the at least one aperture and an in vivo portion of tissue when the at least one suture needle is perpendicular to the elongate plate;
iii) a suture catch on the at least one suture needle configured for releasably catching a portion of the suture during the passage of the at least one suture needle; and
iv) a suture, which prior to being caught by the suture catch, is positioned along a surface of the in vivo tissue.

76. A suturing assembly for suturing in vivo tissue, the suturing assembly comprising:
i) an elongate plate configured for contacting an in vivo tissue;
ii) at least one suture needle moveably connected to the elongate plate;
iii) at least one suture needle moving assembly operatively associated with the at least one suture needle and configured to move the at least one suture needle with respect to the elongate plate; and
iv) a suture catch projecting radially outward from at least one side of the at least one suture needle, the suture catch configured to releasably catch a suture, which prior to being releasably caught by the suture needle, is positioned along a surface of the in vivo tissue.

77. A method of in vivo suturing, comprising:
i) providing at least one suture needle;
ii) providing a suture catch on the at least one suture needle;
iii) causing the at least one suture needle to linearly pass a first pass through a first in vivo tissue surface and a second in vivo tissue surface on an in vivo tissue portion;
iv) catching a first suture portion on the suture catch wherein prior to being caught by the suture catch, the first suture is positioned along a surface of the in vivo tissue; and
v) extending the first suture portion beyond the first in vivo tissue surface in a first loop.

78. The method according to claim 77, wherein following step (v), the method additionally includes:
vi) causing the at least one suture needle to linearly pass a second pass through the first loop; and
vii) passing the at least one suture needle through the first in vivo tissue surface and the second in vivo tissue surface.

79. The method according to claim 78, wherein following step (vii), the method additionally includes:
viii) catching a second suture portion on the suture catch;
ix) extending the second suture portion beyond the first in vivo tissue surface in a second loop; and
x) extending the second loop through the first loop.
